(19) United States
(12) Patent Application Publication
(10) Pub. No.: US 2020/0016084 A1
(43) Pub. Date: Jan. 16, 2020

(54) PARTICLES, COMPOSITIONS, AND METHODS FOR OPHTHALMIC AND/OR OTHER APPLICATIONS

(71) Applicant: Kala pharmaceuticals, Inc., Watertown, MA (US)

(72) Inventor: Winston Zapanta Ong, Stoneham, MA (US)

(21) Appl. No.: 16/502,635

(22) Filed: Jul. 3, 2019

Related U.S. Application Data
(63) Continuation of application No. 62/694,805, filed on Jul. 6, 2018.
(60) Provisional application No. 62/694,819, filed on Jul. 6, 2018.

(51) Int. Cl. A61K 9/50; A61K 31/573; A61K 9/00

(52) U.S. Cl. CPC A61K 9/5031; A61K 9/0048; A61K 31/573

(57) ABSTRACT

This disclosure relates to particles, compositions, and methods that aid particle transport in mucus. The particles, compositions, and methods may be used, in some instances, for ophthalmic and/or other applications.

### CROSS-REFERENCE TO RELATED APPLICATIONS

The present application claims the benefit of U.S. provisional patent application 62/694,819 filed Jul. 6, 2018, and U.S. provisional patent application 62/694,805 filed Jul. 6, 2018, the entire contents of both of which are incorporated by reference herein.

### FIELD

The present disclosure generally relates to particles, compositions, and methods that aid particle transport in mucus. The particles, compositions, and methods may be used in ophthalmic and/or other applications.

### BACKGROUND

A mucus layer present at various points of entry into the body, including the eyes, nose, lungs, gastrointestinal tract, and female reproductive tract, is naturally adhesive and serves to protect the body against pathogens, allergens, and debris by effectively trapping and quickly removing them via mucus turnover. For effective delivery of therapeutic, diagnostic, or imaging particles via mucus membranes, the particles must be able to readily penetrate the mucus layer to avoid mucus adhesion and rapid mucus clearance.

Particles (including microparticles and nanoparticles) that incorporate pharmaceutical agents are particularly useful for ophthalmic applications. However, often it is difficult for administered particles to be delivered to an eye tissue in effective amounts due to rapid clearance and/or other reasons. Accordingly, methods and compositions for administration (e.g., topical application or direct injection) of pharmaceutical agents to the eye would be beneficial.

### SUMMARY

Disclosed herein are pharmaceutical compositions comprising mucus-penetrating particles containing crystalline form I or II of loteprednol etabonate.

Some embodiments include a pharmaceutical composition, comprising particles of loteprednol etabonate crystalline form I or form II; and at least one pharmaceutically acceptable carrier, additive, or diluent.

Some embodiments include a pharmaceutical composition (such as a pharmaceutical composition suitable for administration to an eye), comprising: a plurality of mucus-penetrating coated particles, each coated particle comprising a core particle comprising crystalline form II of loteprednol etabonate; a mucus penetration-enhancing coating comprising a surface-altering agent surrounding the core particle, wherein the surface-altering agent comprises: a) a triblock copolymer comprising a hydrophilic block—hydrophobic block—hydrophilic block configuration, wherein the hydrophobic block has a molecular weight of at least about 2 kDa, and the hydrophilic blocks constitute at least about 15 wt % of the triblock copolymer, the hydrophobic block associates with the surface of the core particle, and the hydrophilic block is present at the surface of the coated particle and renders the coated particle hydrophilic, b) a synthetic polymer having pendant hydroxyl and ester groups in the backbone of the polymer, the polymer having a molecular weight of at least about 1 kDa and less than or equal to about 1000 kDa, wherein the polymer has a degree of hydrolysis of at least about 30% and less than about 95%, or c) a polysorbate; and at least one ophthalmically acceptable carrier, additive, or diluent, wherein the surface altering agent is present on the outer surface of the core particle at a density of at least 0.01 molecules/nm², wherein the surface altering agent is present in the pharmaceutical composition in an amount of between about 0.001% to about 5% by weight in total.

Some embodiments include a pharmaceutical composition (such as a pharmaceutical composition suitable for treating an ocular disorder by administration to an eye), comprising: a plurality of mucus-penetrating coated particles, each coated particle comprising a core particle comprising crystalline form II of loteprednol etabonate and a mucus penetration-enhancing coating comprising a surface-altering agent surrounding the core particle, wherein the surface-altering agent comprises: a) a triblock copolymer comprising a hydrophilic block—hydrophobic block—hydrophilic block configuration, wherein the hydrophobic block has a molecular weight of at least about 2 kDa, and the hydrophilic blocks constitute at least about 15 wt % of the triblock copolymer, b) a synthetic polymer having pendant hydroxyl groups on the backbone of the polymer, the polymer having a molecular weight of at least about 1 kDa and less than or equal to about 1000 kDa, wherein the polymer has a degree of hydrolysis of at least about 30% and less than about 95%, or c) a polysorbate, and at least one ophthalmically acceptable carrier, additive, or diluent, wherein the plurality of coated particles have an average smallest cross-sectional dimension of less than about 1 micron; and wherein the coating on the core particle is present in a sufficient amount to increase the concentration of loteprednol etabonate in a cornea or an aqueous humor after administration to the eye, compared to the concentration of the loteprednol etabonate in the cornea or the aqueous humor when administered as a core particle without the coating.

Also provided herein are methods of treating, diagnosing, preventing, or managing an ocular condition in a subject, the method comprising: administering a pharmaceutical composition as described herein, such as a composition comprising loteprednol etabonate crystalline form I- or II-containing mucus-penetrating particles to an eye of a subject and thereby delivering the loteprednol etabonate to a tissue in the eye of the subject.

As disclosed herein, crystalline form I of loteprednol etabonate has x-ray powder diffraction (XRPD) pattern peaks comprising about 5.6, 7.7, 11.9, 14.1, 17.0 and 18.8±0.2° 2θ. According to some embodiments, the crystalline form I of loteprednol etabonate has additional XRPD peaks at about 16.0, 21.0 and 22.0° 2θ.

As disclosed herein, crystalline form II of loteprednol etabonate has X-ray powder diffraction (XRPD) pattern peaks at about 15.0°, 18.1°, and 19.8°±0.2° 2θ. According to some embodiments, the crystalline form II of loteprednol etabonate has additional XRPD peaks at about 9.8°, 15.6°, 16.6°, 17.2°, 23.0°, 24.8° and 26.3°±0.2° 2θ.

### BRIEF DESCRIPTION OF THE DRAWINGS

FIG. 1 is a schematic drawing of a mucus-penetrating particle having a coating and a core according to one set of embodiments.

FIG. 2 depicts the X-ray powder diffraction (XRPD) pattern of loteprednol etabonate crystalline form I according to one set of embodiments.

FIG. 3 depicts the X-ray powder diffraction (XRPD) pattern of loteprednol etabonate crystalline form II according to one set of embodiments.

### DETAILED DESCRIPTION

A pharmaceutical composition described herein (referred to herein as a “subject composition”) includes a drug-containing particle having a modification to a property of its surface. Although there are a number of surface properties that may be modified, some embodiments relate to surfaces that are modified to provide reduced adhesion to mucus or improved penetration of the particles through physiological mucus, as compared to unmodified drug-containing particles. Thus, disclosed herein are subject compositions comprising mucus-penetrating particles comprising a pharmaceutical composition coated with a mucus penetration-enhancing surface-altering agent.

Particles having efficient transport through mucus barriers may be referred to herein as mucus-penetrating particles (MPPs). The particles may more readily penetrate the mucus layer of a tissue to avoid or minimize mucus adhesion and/or rapid mucus clearance. Therefore, drugs contained in MPPs may be more effectively delivered to, and may be retained longer in, the target issue. As a result, the drugs contained in MPPs may be administered at a lower dose and/or less frequently than formulations lacking mucus penetration-enhancing coatings to achieve similar or superior exposure. Moreover, the relatively low and/or infrequent dosage of the subject compositions may result in fewer or less severe side effects, and/or improved patient compliance.

Non-limiting examples of mucosal tissues include oral (e.g., including the buccal and esophageal membranes and tonsil surface), ophthalmic, gastrointestinal (e.g., including stomach, small intestine, large intestine, colon, and rectum), nasal, respiratory (e.g., including nasal, pharyngeal, tracheal, and bronchial membranes), and genital (e.g., including vaginal, cervical, and urethral membranes) tissues.

Examples of pharmaceutical applications that may benefit from these properties include drug delivery, imaging, and diagnostic applications. For example, a subject composition may be well-suited for ophthalmic applications, and may be used for delivering pharmaceutical agents to the front of the eye, middle of the eye, and/or the back of the eye. With respect to the front of the eye, MPPs may reduce dosage frequency because lower adhesion to mucus may allow the drug to be more evenly spread across the surface of the eye, thereby avoiding the eye's natural clearance mechanisms and prolonging their residence at the ocular surface. Improved mucus penetration allows the drug to penetrate through the mucus coating of the eye more quickly. With respect to the back of the eye, MPPs may allow improved delivery so that a therapeutically effective amount of a drug can reach the back of the eye. In some embodiments, MPPs may effectively penetrate through physiological mucus to facilitate sustained drug release directly to the underlying tissues, as described in more detail below. Mucus-penetrating particles are disclosed in US Patent application publications 2013/0316009 and 2013/01316006, and U.S. Pat. Nos. 9,056,057 and 9,827,191, incorporated by reference herein for all they disclose regarding mucus-penetrating particles.

### Coated Particles

In one aspect, provided herein is a mucus-penetrating particle, comprising a core having an exterior surface, and a mucus penetration-enhancing coating disposed on the exterior surface of the core, wherein:

the core comprises a solid form of loteprednol etabonate, which is crystalline form II of loteprednol etabonate;

the mucus penetration-enhancing coating comprises poloxamer 407; and

the ratio of the total weight of the solid form of loteprednol etabonate to the total weight of the poloxamer 407 is 2:1.

In some embodiments, crystalline form I of loteprednol etabonate has an XRPD pattern comprising a peak, in terms of 2-theta, at about 5.6°, 7.7°, 11.9°, 14.1°, 16.0°, 17.0°, 18.8°, 21.0° and 22.0° or a combination of two or more peaks thereof.

In some embodiments, crystalline form I of loteprednol etabonate has an XRPD pattern comprising peaks, in terms of 2-theta, at about 5.6°, 7.7°, 11.9°, 14.1°, 16.0°, 17.0°, 18.8°, 21.0° and 22.0°.

In some embodiments, crystalline form I of loteprednol etabonate has an XRPD pattern comprising peaks, in terms of 2-theta, at about 5.6°, 7.7°, 11.9°, 14.1°, 17.0° and 18.8°.

In some embodiments, crystalline form I of loteprednol etabonate has an XRPD pattern comprising peaks, in terms of 2-theta, at about 5.6° and 14.1°.

In some embodiments, crystalline form I of loteprednol etabonate has an XRPD pattern comprising peaks, in terms of 2-theta, at about 7.7° and 18.8°.

In some embodiments, crystalline form I of loteprednol etabonate has an XRPD pattern comprising peaks, in terms of 2-theta, at about 11.9° and 17.0°.

In some embodiments, crystalline form I of loteprednol etabonate has an XRPD pattern comprising peaks, in terms of 2-theta, at about 16.0°, 21.0°, and 22.0°.

In some embodiments, crystalline form II of loteprednol etabonate has an XRPD comprising a peak, in terms of 2-theta, at about 9.8°, 15.6°, 16.6°, 17.2°, 23.0°, 24.8°, or 26.3°, or a combination of two or more peaks thereof.

In some embodiments, crystalline form II of loteprednol etabonate has an XRPD pattern comprising peaks, in terms of 2-theta, at about 15.0°, 18.1°, and 19.8°. In some embodiments, crystalline form II of loteprednol etabonate has an XRPD pattern comprising peaks, in terms of 2-theta, at about 9.8°, 15.0°, 15.6°, 16.6°, 17.2°, 18.1°, 19.8°, 23.0°, 24.8°, and 26.3°.

In some embodiments, crystalline form II of loteprednol etabonate has an XRPD pattern comprising peaks, in terms of 2-theta, at about 15.0° and 18.1°.

In some embodiments, crystalline form II of loteprednol etabonate has an XRPD pattern comprising peaks, in terms of 2-theta, at about 15.0°, 18.1°, and 19.8°.

In one aspect, provided herein is a mucus-penetrating particle, comprising a core having an exterior surface, and a mucus penetration-enhancing coating disposed on the exterior surface of the core, wherein:

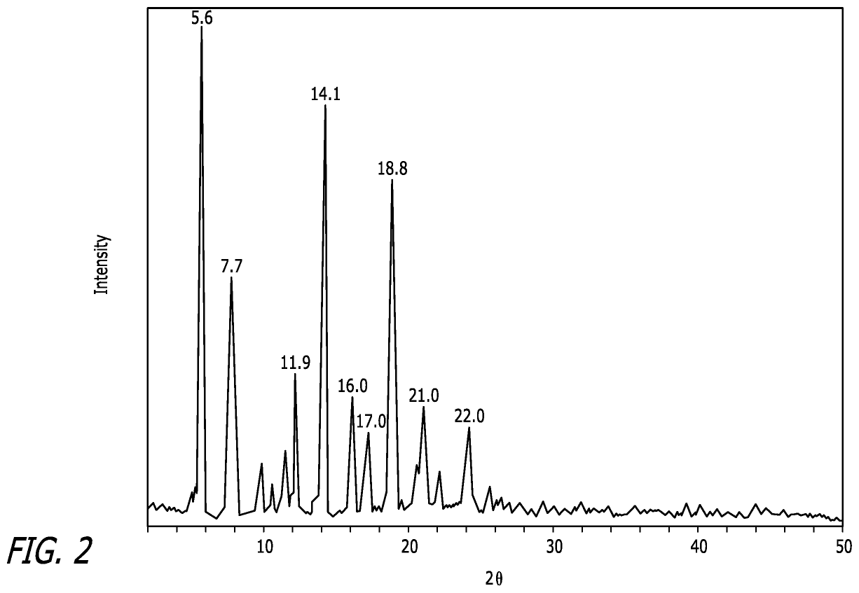

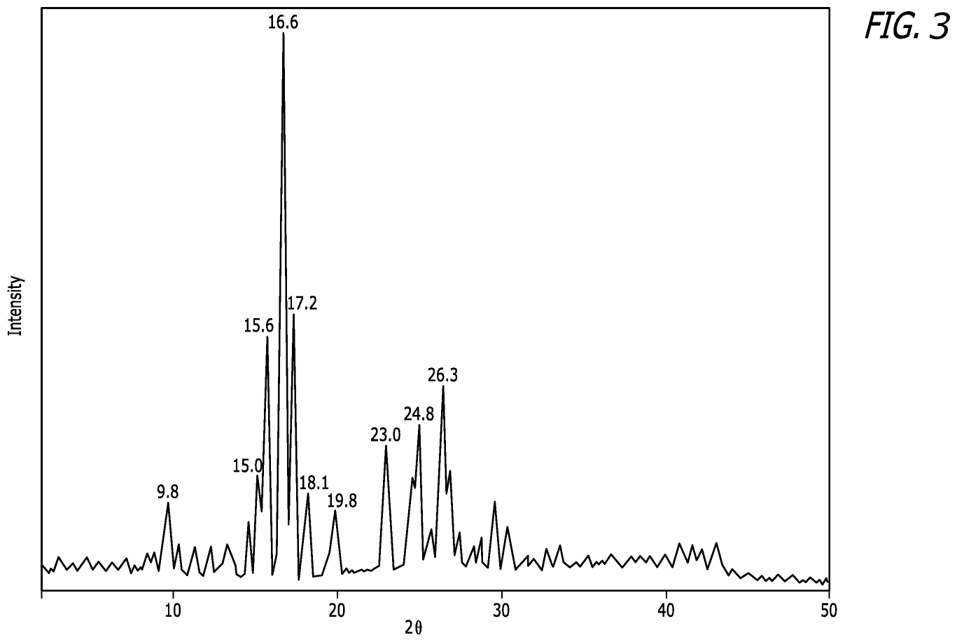

the core comprises a solid form of loteprednol etabonate having an XRPD pattern substantially as shown in FIG. 2 or FIG. 3;

the mucus penetration-enhancing coating comprises poloxamer 407; and

the ratio of the total weight of the solid form of loteprednol etabonate to the total weight of the poloxamer 407 is 2:1.

In another aspect, provided herein is a mucus-penetrating particle, comprising a core having an exterior surface, and a mucus penetration-enhancing coating disposed on the exterior surface of the core, wherein:

the core comprises a solid form of loteprednol etabonate having an XRPD pattern substantially as shown in FIG. 2 or FIG. 3;

the mucus penetration-enhancing coating comprises poloxamer 407;

the solid form of loteprednol etabonate is 1.0% of the pharmaceutical composition by weight in total; and

the ratio of the total weight of the solid form of loteprednol etabonate to the total weight of the poloxamer 407 is 2:1.

In yet another aspect, provided herein is a pharmaceutical composition, comprising a mucus-penetrating coated particle, or a plurality thereof, disclosed herein, and a pharmaceutically acceptable carrier, additive, or diluent, wherein:

the particle is suitable for administration to an eye; and

the mucus penetration-enhancing coating is mucus penetration-enhancing.

In some embodiments of these aspects, poloxamer 407 is between about 0.001% to about 5% of the pharmaceutical composition by weight in total. In some embodiments, crystalline form I or II of loteprednol etabonate is between about 0.00001% and about 10% of the pharmaceutical composition by weight in total.

In some embodiments, the particles described herein have a core-shell type arrangement. The core may comprise any suitable material such as a solid pharmaceutical agent having a relatively low aqueous solubility, a polymeric carrier, a lipid, and/or a protein. The core may also comprise a gel or a liquid in some embodiments. The core may be coated with a coating or shell comprising a mucus penetration-enhancing surface-altering agent that facilitates mobility of the particle in mucus. As described in more detail below, in some embodiments the mucus penetration-enhancing surface-altering agent may comprise a polymer (e.g., a synthetic or a natural polymer) having pendant hydroxyl groups on the backbone of the polymer. The molecular weight and/or degree of hydrolysis of the polymer may be chosen to impart certain transport characteristics to the particles, such as increased transport through mucus. In certain embodiments, the mucus penetration-enhancing surface-altering agent may comprise a triblock copolymer comprising a hydrophilic block—hydrophobic block—hydrophilic block configuration. The molecular weights of each of the blocks may be chosen to impart certain transport characteristics to the particles, such as increased transport through mucus. In certain embodiments, the mucus penetration-enhancing surface-altering agent may comprise a polysorbate.

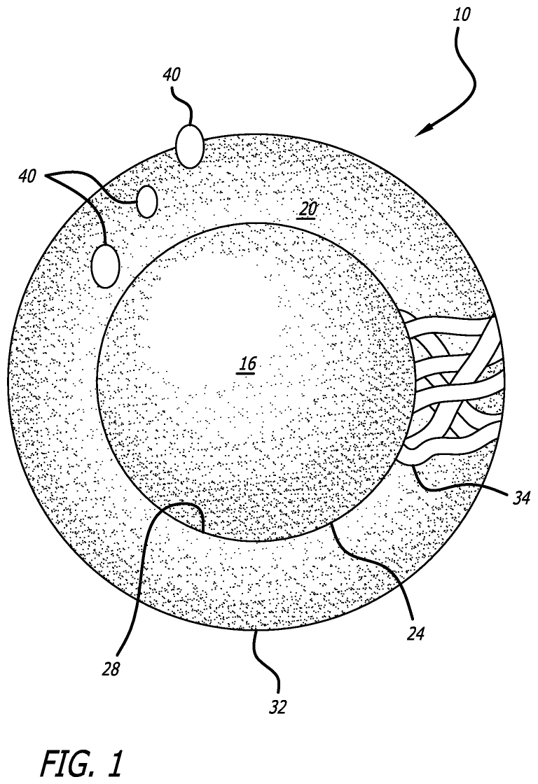

Some embodiments of a coated particle are depicted in FIG. 1. In FIG. 1, particle 10 includes a core 16 (which may be in the form of a particle) and a coating 20 surrounding the core. The core includes a surface 24 to which one or more surface-altering agents can be attached or adhered. For instance, in some cases, core 16 is surrounded by coating 20, which includes an inner surface 28 and an outer surface 32. The coating may comprise one or more surface-altering agents 34, such as a polymer (e.g., a block copolymer and/or a polymer having pendant hydroxyl groups), which may associate with surface 24 of the core. Particle 10 may optionally include one or more components 40 such as targeting moieties, proteins, nucleic acids, and bioactive agents which may optionally impart specificity to the particle. For example, a targeting agent or molecule (e.g., a protein, nucleic acid, nucleic acid analog, carbohydrate, or small molecule), if present, may aid in directing the particle to a specific location in the subject's body. The location may be, for example, a tissue, a particular cell type, or a subcellular compartment. One or more components 40, if present, may be associated with the core, the coating, or both; e.g., they may be associated with surface 24 of the core, inner surface 28 of the coating, outer surface 32 of the coating, and/or embedded in the coating. The one or more components 40 may be associated through covalent bonds, absorption, or attached through ionic interactions, hydrophobic and/or hydrophilic interactions, electrostatic interactions, van der Waals interactions, or combinations thereof. In some embodiments, a component may be attached (e.g., covalently) to one or more of the surface-altering agents of the coated particle.

In certain embodiments, a particle described herein has certain a relative velocity, <Vₘₑₐₙ>ᵣₑₗ, which is defined as follows:

$${〈 {V}_{mean} 〉}_{rel} = \frac{{〈 {V}_{mean} 〉}_{Sample} - {〈 {V}_{mean} 〉}_{Negative control}}{{〈 {V}_{mean} 〉}_{Positive control} - {〈 {V}_{mean} 〉}_{Negative control}} \qquad ( Equation 1 )$$

where <Vₘₑₐₙ> is the ensemble average trajectory-mean velocity, Vₘₑₐₙ is the velocity of an individual particle averaged over its trajectory, the sample is the particle of interest, the negative control is a 200 nm carboxylated polystyrene particle, and the positive control is a 200 nm polystyrene particle densely PEGylated with 2 kDa-5 kDa PEG.

The relative velocity can be measured by a multiple particle tracking technique. For instance, a fluorescent microscope equipped with a CCD camera can be used to capture 15 sec movies at a temporal resolution of 66.7 msec (15 frames/sec) under 100× magnification from several areas within each sample for each type of particles: sample, negative control, and positive control. The sample, negative, and positive controls may be fluorescent particles to observe tracking. Alternatively non-fluorescent particles may be coated with a fluorescent molecule, a fluorescently tagged surface agent, or a fluorescently tagged polymer. An advanced image processing software (e.g., Image Pro or MetaMorph) can be used to measure individual trajectories of multiple particles over a time-scale of at least 3.335 sec (50 frames).

In some embodiments, a MPP described herein has a relative velocity, or a mean relative velocity, in mucus, of at least about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0; up to about 10.0, about 8.0, about 6.0, about 4.0, about 3.0, about 2.0, about 1.9, about 1.8, about 1.7, about 1.6, about 1.5, about 1.4, about 1.3, about 1.2, about 1.1, about 1.0, about 0.9, about 0.8, or about 1.7; about 0.5-6.0, or any velocity in a range bounded by any of these values.

In certain embodiments, an MPP described herein can diffuse through mucus or a mucosal barrier at a greater rate or diffusivity, or may have a greater geometric mean squared displacement, than a control particle or a corresponding particle (e.g., a corresponding particle that is unmodified and/or is not coated with a coating described herein). In some cases, a particle described herein may pass through mucus or a mucosal barrier at a rate of diffusivity, or with a geometric mean squared displacement, that is at least about 10 times, 20 times, 30 times, 50 times, 100 times, 200 times, 500 times, 1000 times, 2000 times, 5000 times, 10000 times, or more; up to about 10000 times, about 5000 times, about 2000 times, about 1000 times, about 500 times, about 200 times, about 100 times, about 50 times, about 30 times, about 20 times, about 10 times; about 10-1000 times higher than a control particle or a corresponding particle; or may have any increase in diffusivity in a range bounded by any of these values.

In some embodiments, an MPP described herein diffuses through a mucosal barrier at a rate approaching the rate or diffusivity at which the particles can diffuse through water. In some cases, a particle described herein may pass through a mucosal barrier at a rate or diffusivity that is at least about 1/10,000, about 1/5000, about 1/2000, about 1/1000, about 1/900, about 1/800, about 1/700, about 1/600, about 1/500, about 1/400, about 1/300, about 1/200, or about 1/100; up to about 1/100, about 1/200, about 1/300, about 1/400, about 1/500, about 1/600, about 1/700, about 1/800, about 1/900, about 1/1000, about 1/2000, about 1/5000, about 1/10; or 1/5000- 1/500, the diffusivity that the particle diffuses through water under identical conditions, or any rate or diffusivity in a range bounded by any of these values.

In a particular embodiment, an MPP described herein may diffuse through human mucus at a diffusivity that is less than about 1/500 the diffusivity that the particle diffuses through water. In some cases, the measurement is based on a time scale of about 1 second, or about 0.5 second, or about 2 seconds, or about 5 seconds, or about 10 seconds.

In certain embodiments provided herein, particles travel through mucus at certain absolute diffusivities. For example, the MPPs described herein may travel at diffusivities of at least about 1×10⁻⁴ μm/s, 2×10⁻⁴ μm/s, 5×10⁻⁴ μm/s, 1×10⁻³ μm/s, 2×10⁻³ μm/s, 5×10⁻³ μm/s, 1×10⁻² μm/s, 2×10⁻² μm/s, 4×10⁻² μm/s, 5×10⁻² μm/s, 6×10⁻² μm/s, 8×10⁻² μm/s, 1×10⁻¹ μm/s, 2×10⁻¹ μm/s, 5×10⁻¹ μm/s, 1 μm/s, or 2 μm/s; up to about 2 μm/s, about 1 μm/s, about 5×10⁻¹ μm/s, about 2×10⁻¹ μm/s, about 1×10⁻¹ μm/s, about 8×10⁻² μm/s, about 6×10⁻² μm/s, about 5×10⁻² μm/s, about 4×10⁻² μm/s, about 2×10⁻² μm/s, about 1×10⁻² μm/s, about 5×10⁻³ μm/s, about 2×10⁻³ μm/s, about 1×10⁻³ μm/s, about 5×10⁻⁴ μm/s, about 2×10⁻⁴ μm/s, or about 1×10⁻⁴ μm/s; or about 2×10⁻⁴-1×10⁻¹ μm/s, or any absolute diffusivity in a range bounded by any of these values. In some cases, the measurement is based on a time scale of about 1 second, or about 0.5 second, or about 2 seconds, or about 5 seconds, or about 10 seconds.

In some embodiments, a subject composition comprises a plurality of particles coated with a mucus penetration-enhancing coating comprising a surface-altering agent, such as a plurality of coated particles. Such a coated particle contains a core comprising the drug and a coating comprising a surface-altering agent.

The surface-altered particles, such as the coated particles described herein, may have any suitable shape and/or size. In some embodiments, a coated particle has a shape substantially similar to the shape of the core. In some cases, a coated particle described herein may be a nanoparticle, i.e., the particle has a characteristic dimension of less than about 1 micrometer, where the characteristic dimension of the particle is the diameter of a perfect sphere having the same volume as the particle. In other embodiments, larger sizes are possible. A plurality of particles, in some embodiments, may also be characterized by an average size, an average characteristic dimension, an average largest cross-sectional dimension, or an average smallest cross-sectional dimension of less than or equal to about 10 μm, less than or equal to about 5 μm, less than or equal to about 1 μm, about 700-800 nm, about 500-700 nm, about 400-500 nm, about 300-400 nm, about 200-300 nm, about 50-200 nm, about 5-100 nm, about 50-75 nm, about 5-50 nm, about 5-40 nm, about 5-35 nm, about 5-30 nm, about 5-25 nm, about 5-20 nm, about 5-15 nm, about 0.1-5 nm, about 200-400 nm, about 200-500 nm, about 100-400 nm, or about 100-300 nm; at least about 5 nm, at least about 20 nm, at least about 50 nm, about 100-700 nm, about 200-500 nm, about 5 μm, about 10 nm, about 1 μm, about 10 nm-5 μm, about 50-500 nm, about 200-500 nm, about 1-10 μm or any size in a range bounded by any of these values. In some embodiments, the sizes of the cores formed by a process described herein have a Gaussian-type distribution.

It is appreciated in the art that the ionic strength of a formulation comprising particles may affect the polydispersity of the particles. Polydispersity is a measure of the heterogeneity of sizes of particles in a formulation. Heterogeneity of particle sizes may be due to differences in individual particle sizes and/or to the presence of aggregation in the formulation. A formulation comprising particles is considered substantially homogeneous or “monodisperse” if the particles have essentially the same size, shape, and/or mass. A formulation comprising particles of various sizes, shapes, and/or masses is deemed heterogeneous or “polydisperse”.

In some embodiments, the polydispersity index of a subject composition, such as a polydispersity index of a particle size or a molecular weight, is at least about 0.005, about 0.01, about 0.05, about 0.1, about 0.15, about 0.2, about 0.3, about 0.4, about 0.5, about 0.6, about 0.7, about 0.8, about 0.9, or about 1; up to about 1, about 0.9, about 0.8, about 0.7, about 0.6, about 0.5, about 0.4, about 0.3, about 0.2, about 0.15, about 0.1, about 0.05, about 0.01, or about 0.005; about 0.1-0.5, about 0.1, about 0.15, about 0.2, or any polydispersity index in a range bounded by any of these values. Polydispersity index may be determined according to ISO standards ISO 13321:1996 E and ISO 22412:2008.

Although many methods for determining sizes of particles are known, the sizes described herein (e.g., average particle sizes, thicknesses) refer to ones measured by dynamic light scattering.

The MPPs may result in a subject composition that is capable of sustaining a therapeutically effective level, or delivering a therapeutically effect amount, of a pharmaceutical agent, such as loteprednol etabonate, in a target tissue. For example, an ophthalmically effective level or an ophthalmically effective amount of the drug-containing MPP may be delivered to an ocular tissue, e.g. an anterior ocular tissue, such as a palpebral conjunctiva, a bulbar conjunctiva, a fornix conjunctiva, an aqueous humor, an anterior sclera, a cornea, an iris, or a ciliary body; or the back of the eye, e.g. a vitreous humor, a vitreous chamber, such as a retina, a macula, a choroid, a posterior sclera, a uvea, an optic nerve, or the blood vessels or nerves which vascularize or innervate a posterior ocular region or site. In some embodiments, the concentration of the pharmaceutical agent, such as loteprednol etabonate, in the tissue may be increased by at least about 10%, about 20%, about 30%, about 40%, about 50%, about 60% or more, within a short relatively amount of time, compared to the concentration of the pharmaceutical agent when administered without the mucus penetration-enhancing coating.

A subject composition may increase the drug level in a target tissue, e.g. the loteprednol etabonate level, within a relatively short amount of time, such as within about 24 hours, about 18 hours, about 12 hours, about 9 hours, about 6 hours, about 4 hours, about 3 hours, about 2 hours, about 1 hour, about 30 minutes, about 20 minutes, about 10 minutes, about 10 minutes to about 2 hours, or any time in a range bounded by any of these values.

A subject composition may achieve a therapeutically effective level or an ophthalmically effective level of loteprednol etabonate in a target tissue, potentially as a result of the mucus penetration-enhancing coating on the MPP, for a sustained period of time after administration, such as least: 10 minutes, 20 minutes, 30 minutes, 1 hour, 2 hours, 4 hours, 6 hours, 9 hours, 12 hours, 18 hours, 1 day, 2 days, 3 days, 4 days, 5 days, 6 days, or 1 week; up to: 1 week, 6 days, 5 days, 4 days, 3 days, 2 days, 1 day, 12 hours, 9 hours, 6 hours, 4 hours, 2 hours, 1 hour; or about 4 hours to about 1 week, about 10 minutes to about 2 hours, or any time in a range bounded by any of these values.

### Particle Core

The core may contain particles of pharmaceutical agents that have a low aqueous solubility, such as a crystalline form of loteprednol etabonate disclosed herein and in CN106279324 and CN106279325, which are incorporated herein by reference for all they disclose regarding loteprednol etabonate crystal forms.

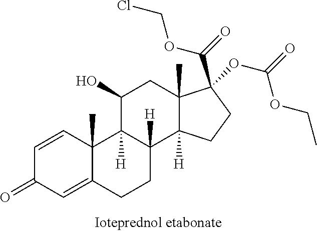

Crystalline form I of loteprednol etabonate comprises XRPD peaks at about 5.6, 7.7, 11.9, 14.1, 17.0 and 18.8±0.2° 2θ. According to some embodiments, the crystalline form I of loteprednol etabonate comprises XRPD peaks at about 5.6, 7.7, 11.9, 14.1, 16.0, 17.0, 18.8, 21.0 and 22.0±0.2° 2θ. The XRPD pattern of loteprednol etabonate crystalline form I can be found in FIG. 2. In some embodiments, loteprednol etabonate has an XRPD pattern as shown in FIG. 2.

Crystalline form II of loteprednol etabonate comprises XRPD peaks at about 15.0°, 18.1°, and 19.8°±0.2° 2θ. According to some embodiments, the crystalline form II of loteprednol etabonate further comprises XRPD peaks at about 9.8°, 15.6°, 16.6°, 17.2°, 23.0°, 24.8°, and 26.3°±0.2° 2θ. The XRPD pattern of loteprednol etabonate crystalline form II can be found in FIG. 3. In some embodiments, loteprednol etabonate has an XRPD pattern as shown in FIG. 3.

The core may comprise the pharmaceutical agent, such as loteprednol etabonate crystalline form I or II. The core may be substantially all pharmaceutical agent, or may comprise additional components, such as a polymer, a lipid, a protein, a gel, a liquid, a surfactant, a tonicity agent (such as glycerin), a buffer, a salt (such as NaCl), a preservative (such as benzalkonium chloride), a chelating agent (such as EDTA), a filler, etc. In some embodiments, the core particles comprise loteprednol etabonate crystalline form I or II that is encapsulated in a polymer, a lipid, a protein, or a combination thereof. In various embodiments, the term encapsulation encompasses any or all of a coating or shell of the encapsulating substance surrounding the rest of the core particle, a solidified co-solution comprising the encapsulating substance and the loteprednol etabonate crystalline form I or II of the core particle, a dispersion of the loteprednol etabonate crystalline form I or II within a matrix comprising the encapsulating substance, and the like.

In embodiments in which the core particles comprise relatively high amounts of loteprednol etabonate crystalline form I or II disclosed herein (e.g., at least about 50 wt % of the core particle), the core particles generally have an increased loading of loteprednol etabonate crystalline form I or II compared to particles that are formed by encapsulating agents into polymeric carriers. This is an advantage for drug delivery applications, since higher drug loadings mean that fewer numbers of particles may be needed to achieve a desired effect compared to the use of particles containing polymeric carriers.

Suitable polymers for use in a core may include a synthetic polymer, e.g. non-degradable polymers such as polymethacrylate and degradable polymers such as polylactic acid, polyethylene glycol, polyglycolic acid and copolymers thereof (such as PLA-PEG), and/or a natural polymer, such as hyaluronic acid, chitosan, and collagen, or a mixture of polymers.

A core may comprise a biodegradable polymer such as poly(ethylene glycol)-poly(propylene oxide)-poly(ethylene glycol) triblock copolymers, poly(lactide) (or poly(lactic acid)), poly(glycolide) (or poly(glycolic acid)), poly(orthoesters), poly(caprolactones), polylysine, poly(ethylene imine), poly(acrylic acid), poly(urethanes), poly(anhydrides), poly(esters), poly(trimethylene carbonate), poly(ethyleneimine), poly(acrylic acid), poly(urethane), poly(beta amino esters) or the like, and combinations, copolymers or derivatives of these and/or other polymers, for example, poly(lactide-co-glycolide) (PLGA).

In certain embodiments, a polymer may biodegrade within a period that is acceptable in the desired application. In certain embodiments, such as in vivo therapy, such degradation occurs in a period usually less than about five years, about one year, about six months, about three months, about one month, about fifteen days, about five days, about three days, or even about one day or less (e.g., about 1-4 hours, about 4-8 hours, about 4-24 hours, about 1-24 hours) on exposure to a physiological solution with a pH between 6 and 8 having a temperature of between 25° C. and 37° C. In some embodiments, the polymer degrades in a period of between about one hour and several weeks.

The pharmaceutical agent may be present in the core in any suitable amount, e.g., at about 1-100 wt %, about 5-100 wt %, about 10-100 wt %, about 20-100 wt %, about 30-100 wt %, about 40-100 wt %, about 50-100 wt %, about 60-100 wt %, about 70-100 wt %, about 80-100 wt %, about 85-100 wt %, about 90-100 wt %, about 95-100 wt %, about 99-100 wt %, about 50-90 wt %, about 60-90 wt %, about 70-90 wt %, about 80-90 wt %, about 85-90 wt % of the core, about 70 wt %, about 75 wt %, about 80 wt %, about 85 wt %, about 90 wt %, about 95 wt %, about 97 wt %, about 98 wt %, about 99 wt %, or about 100 wt %, or any amount in a range bounded by any of these values.

If a polymer is present in the core, the polymer may be present in the core in any suitable amount, e.g., 1-20%, 20-40%, 40-60%, 60-80%, or 80-95% by weight, or any amount in a range bounded by any of those values. In one set of embodiments, the core is substantially free of a polymeric component.

The core may have any suitable shape and/or size. For instance, the core may be substantially spherical, non-spherical, oval, rod-shaped, pyramidal, cube-like, disk-shaped, wire-like, or irregularly shaped. The core may have a largest or smallest cross-sectional dimension of, for example, less than or equal to: about 10 μm, about 5 μm, about 1 μm, about 5-800 nm, about 5-700 nm, about 5-500 nm, about 400 nm, or about 300 nm; 5-200 nm, 5-100 nm, 5-75 nm, 5-50 nm, 5-40 nm, 5-35 nm, 5-30 nm, 5-25 nm, 5-20 nm, 5-15 nm, about 50-500 nm, at least: about 20 nm, about 50 nm, about 100 nm, about 200 nm, about 300 nm, about 400 nm, at least about 500 nm, about 1 μm, or about 5 μm, or any size in a range bounded by any of these values. In some embodiments, the sizes of the cores formed by a process described herein have a Gaussian-type distribution.

### Mucus-Penetration Enhancing Coatings

The surface of a core may be partially or completely covered by a mucus penetration-enhancing coating. The coating may comprise a surface-altering agent, which may be any agent that modifies the surface of the core particles to reduce the adhesion of the particles to mucus and/or to facilitate penetration of the particles through physiological mucus.

In some embodiments, hydrophobic portions of a mucus penetration-enhancing surface-altering agent (e.g., non-hydrolyzed portions of polyvinyl alcohol, hydrophobic polyalkylene oxide, etc.) may allow the polymer to be adhered to the core surface (e.g., in the case of the core surface being hydrophobic), thus allowing for a strong association between the core and the polymer.

In some embodiments, hydrophilic portions of a surface-altering agent (e.g. hydrolyzed potions of polyvinyl alcohol, polethylene oxide, etc.) can render the surface-altering agent, and as a result the particle, hydrophilic. The hydrophilicity may shield the coated particles from adhesive interactions with mucus, which may help to improve mucus transport or penetration.

Examples of suitable surface-altering agents include a block copolymer having one or more relatively hydrophilic blocks and one or more relatively hydrophobic blocks, such as a triblock copolymer, wherein the triblock copolymer comprises a hydrophilic block—hydrophobic block—hydrophilic block configuration; a diblock copolymer having a hydrophilic block—hydrophobic block configuration; a combination of a block copolymer with one or more other polymers suitable for use in a coating; a polymer-like molecule having a nonlinear block configurations, such as nonlinear configurations of combinations of hydrophilic and hydrophobic blocs, such as a comb, a brush, or a star copolymer; a synthetic polymer having pendant hydroxyl groups on the backbone of the polymer; a polysorbate; a surfactant; etc.

The surface-altering agent may have any suitable molecular weight, such as at least about 1 kDa, about 2 kDa, about 4 kDa, about 5 kDa, about 8 kDa, about 9 kDa, about 10 kDa, about 12 kDa, about 15 kDa about 20 kDa, about 25 kDa, about 30 kDa, about 40 kDa, about 50 kDa, about 60 kDa, about 70 kDa, about 80 kDa, about 90 kDa, about 100 kDa about 110 kDa, about 120 kDa, about 130 kDa, about 140 kDa, about 150 kDa, about 200 kDa, about 500 kDa, or about 1000 kDa; less than or equal to about 1000 kDa, about 500 kDa, about 200 kDa, about 180 kDa, about 150 kDa, about 130 kDa, about 120 kDa, about 100 kDa, about 85 kDa, about 70 kDa, about 65 kDa, about 60 kDa, about 50 kDa, about 40 kDa, about 30 kDa, about 20 kDa, about 15 kDa, about 10 kDa; about 10-30 kDa, about 1-100 kDa, about 1-50 kDa, about 1-3 kDa, about 2-7 kDa, about 5-10 kDa, about 8-12 kDa, about 9-15 kDa, about 10-15 kDa, about 12-17 kDa, about 15-25 kDa about 20-30 kDa, about 25-40 kDa, about 30-50 kDa, about 40-60 kDa, about 50-70 kDa; or a molecular weight in a range bounded by any of these values.

When the surface-altering agent is a block copolymer, the molecular weight of the hydrophilic blocks and the hydrophobic blocks of the block copolymers, or the relative amount of the hydrophobic block with respect to the hydrophilic block, may affect the mucoadhesion and/or mucus penetration of a core and association of the block copolymer with the core. Many block copolymers comprise a polyether portion, such as a polyalkylether portion. A polyether block may be relatively hydrophilic (e.g. polyethylene glycol) or relatively hydrophobic (e.g. polyalkylene glycols based upon monomer or repeating units having three or more carbon atoms).

The copolymer may have any suitable molecular weight, such as at least about 1 kDa, about 2 kDa, about 4 kDa, about 5 kDa, about 8 kDa, about 9 kDa, about 10 kDa, about 12 kDa, about 15 kDa about 20 kDa, about 25 kDa, about 30 kDa, about 40 kDa, about 50 kDa, about 60 kDa, about 70 kDa, about 80 kDa, about 90 kDa, about 100 kDa about 110 kDa, about 120 kDa, about 130 kDa, about 140 kDa, about 150 kDa, about 200 kDa, about 500 kDa, or about 1000 kDa; less than or equal to about 1000 kDa, about 500 kDa, about 200 kDa, about 180 kDa, about 150 kDa, about 130 kDa, about 120 kDa, about 100 kDa, about 85 kDa, about 70 kDa, about 65 kDa, about 60 kDa, about 50 kDa, about 40 kDa, about 30 kDa, about 20 kDa, about 15 kDa, about 10 kDa; about 10-30 kDa, about 1-100 kDa, about 1-50 kDa, about 1-3 kDa, about 2-7 kDa, about 5-10 kDa, about 8-12 kDa, about 9-15 kDa, about 10-15 kDa, about 12-17 kDa, about 15-25 kDa about 20-30 kDa, about 25-40 kDa, about 30-50 kDa, about 40-60 kDa, about 50-70 kDa; or a molecular weight in a range bounded by any of these values.

A hydrophobic block may be any suitable block in a block copolymer that is relatively hydrophobic as compared to another block in the copolymer. The hydrophobic block may be substantially present in the interior of the coating and/or at the surface of the core particle, e.g., to facilitate attachment of the coating to the core. Examples of suitable polymers for use in the hydrophobic block include polyalkylethers having three or more carbon atoms in each repeating unit, such as polypropylene glycol, polybutylene glycol, polypentylene glycol, polyhexylene glycol, etc.; esters of polyvinyl alcohol such as polyvinyl acetate; polyvinyl alcohol having a low degree of hydrolysis, etc.

Any suitable amount of the hydrophobic blocks may be used. For example, the hydrophobic block may be a sufficiently large portion of the polymer to allow the polymer to adhere to the core surface, particularly if the core surface is hydrophobic. In certain embodiments, the molecular weight of the (one or more) relatively hydrophobic blocks of a block copolymer, such as poly(propylene oxide) (PPO), is at least about 0.5 kDa, about 1 kDa, about 2 kDa, about 3 kDa, about 4 kDa, about 5 kDa, about 6 kDa, about 10 kDa, about 12 kDa, about 15 kDa, about 20 kDa, about 50 kDa, about 60 kDa, about 70 kDa, about 80 kDa, about 90 kDa, about 100 kDa about 110 kDa, about 120 kDa, about 130 kDa, about 140 kDa, about 150 kDa, about 200 kDa, about 500 kDa, about 1000 kDa; up to about 1000 kDa, about 500 kDa, about 200 kDa, about 150 kDa, about 140 kDa, about 130 kDa, about 120 kDa, about 110 kDa, about 100 kDa, about 90 kDa, about 80 kDa, about 50 kDa, about 20 kDa, about 15 kDa, about 13 kDa, about 12 kDa, about 10 kDa, about 8 kDa, or about 6 kDa; or about 3-15 kDa, 0.5-5 kDa, 0.5-1 kDa, 1-2 kDa, 2-3 kDa, 2-2.5 kDa, 2.5-3 kDa, 3-8 kDa, 3-3.5 kDa, 3.5-4 kDa, 3-4 kDa, 4-5 kDa, about 0.5-3 kDa, 2.5-3 kDa, 2.7-3 kDa, 2.8-3 kDa, 3-3.3 kDa, 3-3.5 kDa, 3.5-3.7 kDa, 3.5-4 kDa, 5-4.5 kDa, 5-10 kDa, or any molecular weight in a range bounded by any of these values.

A hydrophilic block may be any suitable block in a block copolymer that is relatively hydrophilic as compared to another block in the block copolymer. In some cases, the hydrophilic blocks may be substantially present at the outer surface of the particle. For example, the hydrophilic blocks may form a majority of the outer surface of the coating and may help stabilize the particle in an aqueous solution containing the particle. Examples of suitable polymers for use in the hydrophilic block include polyethylene glycol, or synthetic polymers having hydroxyl pendant groups such as polyvinyl alcohol having a high degree of hydrolysis. Any suitable amount of the hydrophilic block may be used, such as an amount that is sufficiently large to render the coated particle hydrophilic when present at the surface of the particle.

In some embodiments, the combined (one or more) relatively hydrophilic blocks, e.g. PEO or polyvinyl alcohol, or repeat units of a block copolymer constitute at least about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, or about 70 wt %; up to about 90 wt %, about 80 wt %, about 60 wt %, about 50 wt %, or about 40 wt % of the block copolymer; or about 30-80 wt %, about 10-30 wt %, 10-40 wt %, about 30-50 wt %, about 40-80 wt %, about 50-70 wt %, about 70-90 wt %, about 15-80 wt %, about 20-80 wt %, about 25-80 wt %, about 30-80 wt %, of the block copolymer, or any percentage in a range bounded by any of these values.

In some embodiments, the molecular weight of the (one or more) relatively hydrophilic blocks or repeat units, such as poly(ethylene oxide) (PEO) or poly(vinyl alcohol) (PVA), of the block copolymer may be at least about 0.5 kDa, about 1 kDa, about 2 kDa, about 3 kDa, about 4 kDa, about 5 kDa, about 6 kDa, about 10 kDa, about 12 kDa, about 15 kDa, about 20 kDa, or about 50 kDa, about 60 kDa, about 70 kDa, about 80 kDa, about 90 kDa, about 100 kDa about 110 kDa, about 120 kDa, about 130 kDa, about 140 kDa, about 150 kDa, about 200 kDa, about 500 kDa, or about 1000 kDa; up to about 1000 kDa, about 500 kDa, about 200 kDa, about 150 kDa, about 140 kDa, about 130 kDa, about 120 kDa, about 110 kDa, about 100 kDa, about 90 kDa, about 80 kDa, about 50 kDa, about 20 kDa, about 15 kDa, about 13 kDa, about 12 kDa, about 10 kDa, about 8 kDa, about 6 kDa, about 5 kDa, about 3 kDa, about 2 kDa, about 1 kDa; about 1-2 kDa, about 2-4 kDa, about 3-15 kDa, about 4-7 kDa, 7-10 kDa, about 10-12 kDa, about 10-15 kDa, or any molecular weight in a range bounded by any of these values.

In embodiments in which two hydrophilic blocks flank a hydrophobic block, the molecular weights, and the chemical identity, of the two hydrophilic blocks may be substantially the same or different.

In certain embodiments, the polymer is a triblock copolymer of a polyalkyl ether (e.g., polyethylene glycol or polypropylene glycol) and another polymer (e.g., a synthetic polymer having pendant hydroxyl groups on the backbone of the polymer (e.g., PVA). In certain embodiments, the polymer is a triblock copolymer of a polyalkyl ether (such as polyethylene glycol) and another polyalkyl ether. In certain embodiments, the polymer includes a polypropylene glycol unit flanked by two more hydrophilic units. In certain embodiments, the polymer includes two polyethylene glycol units flanking a more hydrophobic unit. The molecular weights of the two blocks flanking the central block may be substantially the same or different.

In certain embodiments, the polymer is of Formula 1:

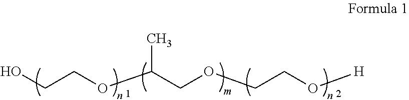

With respect to Formula 1, m is about 2-1730, about 5-70, about 5-100, about 20-100, about 10-20, about 20-30, about 30-40, about 40-50, about 50-60, about 60-70, about 10-50, about 40-60, about 50-70, about 50-about 100, about 100-300, about 300-500, about 500-700, about 700-1000, about 1000-1300, about 1300-1600, about 1600-2000, about 15, about 20, about 31, about 41, about 51, about 61, about 68, or any integer in a range bounded by any of these values.

With respect to Formula 1, n¹ and n² may be the same or different. In some embodiments, n¹+n², is 2-1140, 2-10, 10-30, 30-40, 40-70, 70-150, 150-200, 10-170, 50-150, 90-110, 100-200, 200-400, 400-600, 600-800, 800-1000, 1000-1500, about 2, about 6, about 8, about 9, about 18, about 29, about 35, about 39, about 41, about 68, about 82, about 127, about 164, about 191, or any integer in a range bounded by any of these values. In certain embodiments, n¹+n² is at least 2 times m, 3 times m, or 4 times m.

With respect to Formula 1, in some embodiments m is about 10-30 and n¹+n² is about 2-10, m is about 10-30 and n¹+n² is about 10-30, m is about 30-50 and n¹+n² is about 2-10, m is about 40-60 and n¹+n² is about 2-10, m is about 30-50 and n¹+n² is about 40-100, m is about 60-80 and n¹+n² is about 2-10, m is about 40-60 and n¹+n² is about 20-40, m is about 10-30 and n¹+n² is about 10-30, m is about 60-80 and n¹+n² is about 20-40, m is about 40-60 and n¹+n² is about 40-100, m is about 30-50 and n¹+n² is about 100-200, m is about 30-50 and n¹+n² is about 100-200, m is about 60-80 and n¹+n² is about 100-200, or m is about 60-80 and n¹+n² is about 20-40.

In certain embodiments, the coating includes a surface-altering agent comprising a (poly(ethylene glycol))-(poly(propylene oxide))-(poly(ethylene glycol)) triblock copolymer (hereinafter “PEG-PPO-PEG triblock copolymer”), present in the coating alone or in combination with another polymer such as a synthetic polymer having pendant hydroxyl groups on the backbone of the polymer (e.g., PVA). As described herein, the PEG blocks may be interchanged with PEO blocks in some embodiments. The molecular weights of the PEG (or PEO) and PPO segments of the PEG-PPO-PEG triblock copolymer may be selected so as to reduce the mucoadhesion of the particle, as described herein. Without wishing to be bound by theory, a particle having a coating comprising a PEG-PPO-PEG triblock copolymer may have reduced mucoadhesion as compared to a control particle due to, at least in part, the display of a plurality of PEG (or PEO) segments on the particle surface. The PPO segment may be adhered to the core surface (e.g., in the case of the core surface being hydrophobic), thus allowing for a strong association between the core and the triblock copolymer. In some cases, the PEG-PPO-PEG triblock copolymer is associated with the core through non-covalent interactions. For purposes of comparison, the control particle may be, for example, a carboxylate-modified polystyrene particle of similar size as the coated particle in question.

In some embodiments, a triblock copolymer, such as a PEO-PPO-PEO copolymer, has an average molecular weight that is at least about 1 kDa, about 2 kDa, about 4 kDa, about 5 kDa, about 8 kDa, about 9 kDa, about 10 kDa; less than or equal to about 100 kDa, about 50 kDa, about 20 kDa, about 15 kDa, about 10 kDa; or is about 1-3 kDa, about 1-3 kDa, about 2-4 kDa, about 3-5 kDa, about 4-6 kDa, about 5-7 kDa, about 6-8 kDa, about 7-9 kDa, about 8-10 kDa, about 5-7 kDa, about 2-7 kDa, about 5-10 kDa, about 8-12 kDa, about 9-15 kDa, about 10-15 kDa, about 12-17 kDa, about 15-25 kDa about 20-30 kDa, about 25-40 kDa, about 30-50 kDa, about 40-60 kDa, about 50-70 kDa; or a molecular weight in a range bounded by any of these values.

In certain embodiments, a surface-altering agent includes a polymer comprising a poloxamer having the trade name Pluronic®. Pluronic® polymers that may be useful in the embodiments described herein include, but are not limited to, F127, F38, F108, F68, F77, F87, F88, F98, F123, L101, L121, L31, L35, L43, L44, L61, L62, L64, L81, L92, N3, P103, P104, P105, P123, P65, P84, and P85. In some embodiments, the surface-altering agent comprises Pluronic® F127, F108, P123, P105, or P103.

Examples of molecular weights of certain Pluronic® molecules are shown in Table 1.

TABLE 1
Molecular Weights of Pluronic ® molecules

| | | Average | MW | PEO | MW |
|---|---|---|---|---|---|
| Pluronic ® | Poloxamer | MW | PPO | wt % | PEO |
| L31 | 101 | 1000 | 900 | 10 | 100 |
| L44 | 124 | 2000 | 1200 | 40 | 800 |
| L81 | 231 | 2667 | 2400 | 10 | 267 |
| L101 | 331 | 3333 | 3000 | 10 | 333 |
| P65 | 185 | 3600 | 1800 | 50 | 1800 |
| L121 | 401 | 4000 | 3600 | 10 | 400 |
| P103 | 333 | 4286 | 3000 | 30 | 1286 |
| F38 | 108 | 4500 | 900 | 80 | 3600 |
| P105 | 335 | 6000 | 3000 | 50 | 3000 |
| F87 | 237 | 8000 | 2400 | 70 | 5600 |
| F68 | 188 | 9000 | 1800 | 80 | 7200 |
| F127 | 407 | 12000 | 3600 | 70 | 8400 |
| P123 | 403 | 5750 | 4030 | 30 | 1730 |
| F108 | 338 | 14600 | 3250 | 80 | 11350 |

A surface-altering agent may include a synthetic polymer having pendant hydroxyl groups on the backbone of the polymer, such as a poly(vinyl alcohol), a partially hydrolyzed poly(vinyl acetate), a copolymer of vinyl alcohol and vinyl acetate, a poly(ethylene glycol)-poly(vinyl acetate)-poly(vinyl alcohol) copolymer, a poly(ethylene glycol)-poly(vinyl alcohol) copolymer, a poly(propylene oxide)-poly(vinyl alcohol) copolymer, a poly(vinyl alcohol)-poly(acryl amide) copolymer, etc.

The synthetic polymer described herein (e.g., one having pendant hydroxyl groups on the backbone of the polymer) may have any suitable molecular weight, such as at least about 1 kDa, about 2 kDa, about 5 kDa, about 8 kDa, about 9 kDa, about 10 kDa, about 12 kDa, about 15 kDa about 20 kDa, about 25 kDa, about 30 kDa, about 40 kDa, about 50 kDa, about 60 kDa, about 70 kDa, about 80 kDa, about 90 kDa, about 100 kDa, about 110 kDa, about 120 kDa, about 130 kDa, about 140 kDa, about 150 kDa, about 200 kDa, about 500 kDa, or about 1000 kDa; up to about 1000 kDa, about 500 kDa, about 200 kDa, about 180 kDa, about 150 kDa, about 130 kDa, about 120 kDa, about 100 kDa, about 85 kDa, about 70 kDa, about 65 kDa, about 60 kDa, about 50 kDa, about 40 kDa, about 30 kDa, about 20 kDa, about 15 kDa, or about 10 kDa; about 1-1000 kDa, about 1-10 kDa, about 5-20 kDa, about 10-30 kDa, about 20-40 kDa, about 30-50 kDa, about 40-60 kDa, about 50-70 kDa, about 60-80 kDa, about 70-90 kDa, about 80-100 kDa, about 90-110 kDa, about 100-120 kDa, about 110-130 kDa, about 120-140 kDa, about 130-150 kDa, about 140-160 kDa, about 150-170 kDa, or any molecular weight in a range bounded by any of these values.

Poly(vinyl alcohol) may be prepared by polymerizing a vinyl ester to produce a poly(vinyl ester), such as poly(vinyl acetate), and then hydrolyzing the ester to leave free pendant hydroxy groups. Partially hydrolyzed PVA comprises two types of repeating units: vinyl alcohol units (which are relatively hydrophilic) and residual vinyl acetate units (which are relatively hydrophobic). Some embodiments may include one or more blocks of vinyl alcohol units and one or more blocks of vinyl acetate units. In certain embodiments, the repeat units form a copolymer, e.g., a diblock, triblock, alternating, or random copolymer.

The amount of hydrolysis, or the percentage of vinyl alcohol units as compared to the total number of vinyl alcohol+vinyl acetate units, may affect or determine the relative hydrophilicity or hydrophobicity of a poly(vinyl alcohol), and can affect the mucus penetration of the particles. It may be helpful for the degree of hydrolysis to be low enough to allow sufficient adhesion between the PVA and the core (e.g., in the case of the core being hydrophobic). It may also be helpful for the degree of hydrolysis to be high enough to enhance particle transport in mucus. The appropriate level of hydrolysis may depend on additional factors such as the molecular weight of the polymer, the composition of the core, the hydrophobicity of the core, etc.

Less than 95% hydrolysis in a poly(vinyl alcohol) may render a particle mucus penetrating. In some embodiments, a synthetic polymer (e.g., PVA or partially hydrolyzed poly(vinyl acetate) or a copolymer of vinyl alcohol and vinyl acetate) may have a hydrolysis level of at least: about 30%, about 35%, about 40%, about 45%, about 50%, about 55%, about 60%, about 65%, about 70%, about 75%, about 80%, about 85%, about 87%, about 90%, about 95%, or about 98%; up to about 100%, about 98%, about 97%, about 96%, about 95%, about 94%, about 93%, about 92%, about 91%, about 90%, about 87%, about 85%, about 80%, about 75%, about 70%, or about 60%; about 80-95%, about 30-95%, about 70-94%, about 30-95%, or about 70-94%, or any percentage in a range bounded by any of these values.

In some embodiments, a synthetic polymer described herein is, or comprises, PVA. PVA is a non-ionic polymer with surface active properties. In some embodiments, the hydrophilic units of a synthetic polymer described herein may be substantially present at the outer surface of the particle.

The molar fraction of the relatively hydrophilic units and the relatively hydrophobic units of a synthetic polymer may be selected so as to reduce the mucoadhesion of a core and to ensure sufficient association of the polymer with the core, respectively. The molar fraction of the relatively hydrophilic units to the relatively hydrophobic units of a synthetic polymer may be, for example, 0.5:1 (hydrophilic units:hydrophobic units), 1:1, 2:1, 3:1, 5:1, 7:1, 10:1, 15:1, 20:1, 25:1, 30:1, 40:1, 50:1, 75:1, 100:1; up to 100:1, 75:1, 50:1, 40:1, 30:1, 25:1, 20:1, 15:1, 10:1, 7:1, 5:1, 3:1, 2:1, or 1:1; 2:1-4:1, 3:1-5:1, 4:1-6:1, 5:1-7:1, 6:1-8-1, 7:1-9:1, 8:1-10:1, 9:1-11:1, 10:1-20:1, 15:1-50:1, 20:1-1000:1, or any molar ratio in a range bounded by any of these values.

Examples of PVA polymers having various molecular weights and degree of hydrolysis are shown in Table 2. The molecular weight (MW) and hydrolysis degree values were provided by the manufacturers.

TABLE 2
Exemplary PVAs.

| PVA acronym* | MW, kDa | Hydrolysis degree, % |
|---|---|---|
| 2K75 | 2 | 75-79 |
| 9K80 | 9-10 | 80 |
| 13K87 | 13-23 | 87-89 |
| 13K98 | 13-23 | 98 |
| 31K87 | 31-50 | 87-89 |
| 31K98 | 31-50 | 98-99 |
| 57K86 | 57-60 | 86-89 |
| 85K87 | 85-124 | 87-89 |
| 85K99 | 85-124 | 99+ |
| 95K95 | 95 | 95 |
| 105K80 | 104 | 80 |
| 130K87 | 130 | 87-89 |

*PVA acronym: XXKYY, where XX stands for the PVA's lower-end molecular weight in kDa and YY stands for the PVA's lower-end hydrolysis in %.

In certain embodiments, the synthetic polymer is represented by Formula 2:

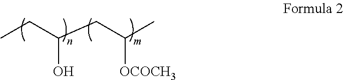

With respect to Formula 2 above, m is 0-11630. Similarly, the value of m may vary. For instance, in certain embodiments, m is at least 5, 10, 20, 30, 50, 70, 100, 150, 200, 250, 300, 350, 400, 500, 800, 1000, 1200, 1500, 1800, 2000, 2200, 2400, 2600, 3000, 5000, 10000, or 15000; up to 15000, 10000, 5000, 3000, 2800, 2400, 2000, 1800, 1500, 1200, 1000, 800, 500, 400, 350, 300, 250, 200, 150, 100, 70, 50, 30, 20, or 10; 5-200, 10-100, 100-150, 150-200, 200-300, 300-400, 400-600, 600-800, 800-1000, 1000-1200, 1200-1400, about 20, about 92, about 102, about 140, about 148, about 247, about 262, about 333, about 354, about 538, about 570, about 611, about 643, about 914, about 972, about 1061, about 1064, about 1333, about 1398, about 1418, or any integer in a range bounded by any of these values.

With respect to Formula 2 above, n is 0-22730. In some embodiments, n is at least 5, 10, 20, 30, 50, 100, 200, 300, 500, 800, 1000, 1200, 1500, 1800, 2000, 2200, 2400, 2600, 3000, 5000, 10000, 15000, 20000, or 25000; up to 30000, 25000, 20000, 25000, 20000, 15000, 10000, 5000, 3000, 2800, 2400, 2000, 1800, 1500, 1200, 1000, 800, 500, 300, 200, 100, or 50; 25-20600, 50-2000, 5-1100, 0-400, 1-400; or 1-10, 10-20, 20-30, 30-50, 50-80, 80-100, 100-150, 150-200, 200-300, about 3, about 5, about 6, about 9, about 10, about 14, about 19, about 23, about 26, about 34, about 45, about 56, about 73, about 87, about 92, about 125, about 182, about 191, about 265, or any integer in a range bounded by any of these values.

It is noted that n and m may represent the total content of the vinyl alcohol and vinyl acetate repeat units in the polymer, or may represent block lengths.

With respect to Formula 2, above, in some embodiments m is about 1-100 and n is about 1-10, m is about 1-100 and n is about 20-30, m is about 100-200 and n is about 20-30, m is about 100-200 and n is about 10-20, m is about 200-300 and n is about 30-50, m is about 100-200 and n is about 1-10, m is about 200-300 and n is about 1-10, m is about 300-500 and n is about 30-50, m is about 500-700 and n is about 70-90, m is about 300-500 and n is about 1-10, m is about 500-700 and n is about 1-10, m is about 500-700 and n is about 70-90, m is about 500-700 and n is about 90-150, m is about 700-100 and n is about 90-150, m is about 1000-1200 and n is about 150-200, m is about 700-100 and n is about 1-10, m is about 1200-1500 and n is about 10-20, m is about 1000-1200 and n is about 50-70, m is about 1000-1200 and n is about 200-300, or m is about 1200-1500 and n is about 150-200.

In some embodiments, the PVA is PVA2K75, PVA9K80, PVA13K87, PVA31K87, PVA57K86, PVA85K87, PVA105K80, or PVA130K87. The PVA acronyms are described using the formula PVAXXKYY, where XX stands for the PVA's lower-end molecular weight in kDa and YY stands for the PVA's lower-end hydrolysis in %.

A surface-altering agent may include a polysorbate. Polysorbates are typically derived from PEGylated sorbitan (a derivative of sorbitol) esterified with fatty adds. Examples of polysorbates include polyoxyethylene sorbitan monooleate (e.g., Tween® 80), polyoxyethylene sorbitan monostearate (e.g., Tween® 60), polyoxyethylene sorbitan monopalmitate (e.g., Tween® 40), and polyoxyethylene sorbitan monolaurate (e.g., Tween® 20).

In some embodiments, the surface-altering agent comprises a poloxamer, a poly(vinyl alcohol), a polysorbate, or a combination thereof.

In some embodiments, the surface-altering agent comprises L-α-phosphatidylcholine (PC), 1,2-dipalmitoylphosphatidycholine (DPPC), oleic acid, sorbitan trioleate, sorbitan mono-oleate, sorbitan monolaurate, a polyoxylene sorbitan fatty acid ester (Tweens), a polysorbate (e.g., polyoxyethylene sorbitan monooleate) (e.g., Tween® 80), polyoxyethylene sorbitan monostearate (e.g., Tween® 60), polyoxyethylene sorbitan monopalmitate (e.g., Tween® 40), polyoxyethylene sorbitan monolaurate (e.g., Tween® 20), natural lecithin, oleyl polyoxyethylene ether, stearyl polyoxyethylene ether, lauryl polyoxyethylene ether, polyoxylene alkyl ethers, a block copolymer of oxyethylene and oxypropylene, apolyoxyethylene stearate, polyoxyethylene castor oil and/or a derivative thereof, a Vitamin-E-PEG or a derivative thereof, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol, cetyl pyridinium chloride, benzalkonium chloride, olive oil, glyceryl monolaurate, corn oil, cotton seed oil, sunflower seed oil, or a derivative and/or combination thereof.

The surface-altering agent may be present in the pharmaceutical composition in any suitable amount, such as an amount between about 0.001-5%, about 0.001-1%, about 1-2%, about 2-3%, about 3-4%, or about 4-5% by weight in total.

The surface-altering agent may be present in any suitable amount with respect to the pharmaceutical agent. In some embodiments, the ratio of surface-altering agent to pharmaceutical agent may be at least about 0.001:1 (weight ratio, molar ratio, or w:v ratio), about 0.01:1, about 0.01:1, about 1:1, about 2:1, about 3:1, about 5:1, about 10:1, about 25:1, about 50:1, about 100:1, or about 500:1. In some embodiments, the ratio of surface-altering agent to pharmaceutical agent is up to about 1000:1 (weight ratio, molar ratio, or w:v ratio), about 500:1, about 100:1, about 75:1, about 50:1, about 25:1, about 10:1, about 5:1, about 3:1, about 2:1, about 1:1, about 0.1:1; and/or about 5:1-50:1, or any ratio in a range bounded by any of these values.

Typically, a coating may be on the surface of, or partially or completely surround or coat, the core. In some embodiments, the surface-altering agent may surround the core particle.

The coating may adhere, or be covalently or non-covalently bound or otherwise attached, to the core. For example, the surface-altering agent may be covalently attached to a core particle, non-covalently attached to a core particle, adsorbed to a core particle, or coupled or attached to the core particle through ionic interactions, hydrophobic and/or hydrophilic interactions, electrostatic interactions, van der Waals interactions, or combinations thereof. A surface-altering agent may be oriented in a particular configuration in the coating of the particle. For example, in some embodiments in which a surface-altering agent is a triblock copolymer, such as a triblock copolymer having a hydrophilic block—hydrophobic block—hydrophilic block configuration, and the hydrophobic block may be oriented towards the surface of the core, and the hydrophilic blocks may be oriented away from the core surface (e.g., towards the exterior of the particle).

The coating may include one layer of material (e.g., a monolayer), or multilayers of materials. A single type of surface-altering agent may be present, or multiple types of surface-altering agent.

The surface-altering agent may be present on the surfaces of the core particles at any density that is effective to reduce adhesion to mucus or improved penetration of the particles through mucus. For example, the surface-altering agent may be present on the surfaces of the core particles at a density of at least: about 0.001, about 0.002, about 0.005, about 0.01, about 0.02, about 0.05, about 0.1, about 0.2, about 0.5, about 1, about 2, about 5, about 10, about 20, about 50, or about 100; up to: about 100, about 50, about 20, about 10, about 5, about 2, about 1, about 0.5, about 0.2, about 0.1, about 0.05, about 0.02, or about 0.01; or about 0.01-1 units or molecules/nm²; or any density in a range bounded by any of these values.

Those of ordinary skill in the art will be aware of methods to estimate the average density of surface-altering moieties on the core particle (see, for example, S. J. Budijono et al., Colloids and Surfaces A: Physicochem. Eng. Aspects 360 (2010) 105-110 and Joshi, et al., Anal. Chim. Acta 104 (1979) 153-160, each of which is incorporated herein by reference). For example, as described herein, the average density of surface-altering moieties can be determined using HPLC quantitation and DLS analysis. A suspension of particles for which surface density determination is of interest is first sized using DLS: a small volume is diluted to an appropriate concentration (˜100 μg/mL, for example), and the z-average diameter is taken as a representative measurement of particle size. The remaining suspension is then divided into two aliquots. Using HPLC, the first aliquot is assayed for the total concentration of core material and for the total concentration of surface-altering moiety. Again using HPLC, the second aliquot is assayed for the concentration of free or unbound surface-altering moiety. In order to get only the free or unbound surface-altering moiety from the second aliquot, the particles, and therefore any bound surface-altering moiety, are removed by ultracentrifugation. By subtracting the concentration of the unbound surface-altering moiety from the total concentration of surface-altering moiety, the concentration of bound surface-altering moiety can be determined. Since the total concentration of core material was also determined from the first aliquot, the mass ratio between the core material and the surface-altering moiety can be determined. Using the molecular weight of the surface-altering moiety the number of surface-altering moiety to mass of core material can be calculated. To turn this number into a surface density measurement, the surface area per mass of core material needs to be calculated. The volume of the particle is approximated as that of a sphere with the diameter obtained from DLS allowing for the calculation of the surface area per mass of core material. In this way the number of surface-altering moieties per surface area can be determined. The surface area of a perfect sphere with the diameter of the core particles can be determined by dynamic light scattering. In alternative embodiments surface area is measured as the Brunauer-Emmett-Teller specific surface area which is based on the adsorption of gas molecules to solid surfaces. Most typically nitrogen is the gas used.

In certain embodiments in which the surface-altering agent is adsorbed onto a surface of a core, the surface-altering agent may be in equilibrium with other molecules of the surface-altering agent in solution. In some cases, the adsorbed surface-altering agent may be present on the surface of the core at a density described herein.

A coating comprising a surface-altering agent may partially or completely surround the core. For example, the coating may surround at least about 10%, at least about 30%, at least about 50%, at least about 60%, at least about 70%, at least about 80%, at least about 90%, at least about 99%, up to about 100%, up to about 90%, up to about 80%, up to about 70%, up to about 60%, or up to about 50%, about 80-100% of the surface area of a core, or any percentage in a range bounded by any of these values.

A coating of a particle can have any suitable thickness. For example, a coating may have an average thickness of at least about 1 nm, about 5 nm, about 10 nm, about 30 nm, about 50 nm, about 100 nm, about 200 nm, about 500 nm, about 1 μm, or about 5 μm. In other embodiments, the coating may have an average thickness of up to about 5 μm, about 1 μm, about 500 nm, about 200 nm, about 100 nm, about 50 nm, about 30 nm, about 10 nm, or about 5 nm. In other embodiments, the coating may have an average thickness of about 1-100 nm, or any thickness in a range bounded by any of the preceding values. Thickness is determined by comparison of particle sizes of the coated particle and the corresponding uncoated core particle using dynamic light scattering.

In some embodiments, two or more surface-altering agents, such as two or more of a PEG-PPO-PEG triblock copolymer, a synthetic polymer having pendant OH groups (e.g. PVA), and a polysorbate, may be present in the coating. Furthermore, although many of the embodiments described herein involve a single coating, in other embodiments, a particle may include more than one coating (e.g., at least two, three, four, five, or more coatings), and each coating need not be formed of, or comprise, a mucus penetrating material. In some cases, an intermediate coating (i.e., a coating between the core surface and an outer coating) may include a polymer that facilitates attachment of an outer coating to the core surface. In many embodiments, an outer coating of a particle includes a polymer comprising a material that facilitates the transport of the particle through mucus.

### Pharmaceutical Formulations

Provided herein are also pharmaceutical compositions comprising LE form I or form II as described herein and a pharmaceutically acceptable carrier, additive, or diluent. The compositions are formulated in accordance with methods known in the art for a particular route of administration desired.

Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

In some embodiments, the pharmaceutical compositions are for local delivery, such as topical formulations. Examples of various types of preparations for topical administration include ointments, lotions, creams, powders, drops (e.g., eye or ear or nose drops), sprays (e.g., for the nose or throat), suppositories, retention enemas, and aerosols.

Ointments and creams can, for example, be formulated with an aqueous or oily base with the addition of suitable thickening and/or gelling agents and/or glycols. Such base can thus, for example, include water and/or an oil such as liquid paraffin or a vegetable oil such as arachis oil or castor oil, or a glycolic solvent such as propylene glycol or 1,3-butanediol. Thickening agents which can be used according to the nature of the base include soft paraffin, aluminum stearate, cetostearyl alcohol, polyethylene glycols, woolfat, hydrogenated lanolin and beeswax and/or glyceryl monostearate and/or non-ionic emulsifying agents.

In some embodiments, the pharmaceutical compositions are in a cream or ointment form. In some certain embodiments, the cream or ointment comprises an aromatic alcohol such as benzyl alcohol, phenylethyl alcohol, or phenoxyethyl alcohol.

Lotions can be formulated with an aqueous or oily base and will in general also include one or more of the following, namely, emulsifying agents, dispersing agents, suspending agents, thickening agents, solvents, coloring agents and perfumes. Powders can be formed with the aid of any suitable powder base e.g., talc, lactose or starch. Drops can be formulated with an aqueous base also comprising one or more dispersing agents, suspending agents or solubilizing agents, etc. Spray compositions can, for example, be formulated as aerosols with the use of a suitable propellant, e.g., dichlorodifluoromethane or trichlorofluoromethane.

Nebulized or powdered formulations can be prepared for oral inhalation in the treatment of asthma, COPD or the like, as is well-known in the art. For example, an inhalation formulation suitable for use in the treatment of asthma can be prepared as a metered-dose aerosol unit according to procedures well-known to those skilled in the art of pharmaceutical formulations. Such an aerosol unit may contain a microcrystalline suspension of a loteprednol etabonate crystalline form as described herein in suitable propellants (e.g., trichlorofluoromethane and dichlorodifluoromethane), with oleic acid or other suitable dispersing agent.

Solutions and suspensions can be prepared for oral or rectal administration for use in the treatment of inflammations of the intestines, for example, as described in more detail in the examples hereinafter. Parenteral/injectable formulations can be prepared for direct injection into the joints in the treatment of arthritis in accord with methods well-known to those skilled in the art of parenteral formulations.

Another example of a pharmaceutical composition is a foam suitable for treatment of a wide variety of inflammatory anorectal disorders, to be applied anally or perianally, comprising a loteprednol etabonate crystalline form as described herein, and 1 a local anesthetic such as pramoxine hydrochloride, in a mucoadhesive foam base of propylene glycol, ethoxylated stearyl alcohol, polyoxyethylene-10-stearyl ether, cetyl alcohol, methyl paraben, propyl paraben, triethanolamine, and water, with inert propellants.

Yet another pharmaceutical formulation is a solution or suspension suitable for use as a retention enema, a single dose of a loteprednol etabonate crystalline form as described herein. In some embodiments, such formulation further comprises sodium chloride, polysorbate 80 and water (the water being added shortly before use). The suspension can be administered as a retention enema or by continuous drip several times weekly in the treatment of ulcerative colitis.

In some embodiments, the pharmaceutical compositions are for topical ophthalmic administration or implantation into the conjunctival sac or anterior chamber of the eye. Accordingly, in some embodiments, the compositions disclosed herein also include ophthalmic compositions that comprises loteprednol form I or form II, and an ophthalmically acceptable carrier, diluent, or excipient. As used herein, the term “ophthalmically acceptable carrier, diluent, or excipient” refers to any material that can contain and release the agent and that is compatible with the eye.

In some embodiments, the ophthalmic composition is a liquid composition. In some embodiments, the ophthalmic composition is a semi-solid composition. In some embodiments, the ophthalmic composition is a topical composition. The topical compositions include, but are not limited to liquid and semi-solid compositions. In some embodiments, the ophthalmic composition is a topical composition. In some embodiments, the topical composition comprises aqueous solution, an aqueous suspension, an ointment or a gel. In some embodiments, the ophthalmic composition is topically applied to the front of the eye, under the upper eyelid, on the lower eyelid and in the cul-de-sac.

The ophthalmic compositions as disclosed herein may also include various other ingredients, including but not limited to surfactants, suspending agents, tonicity agents, buffers, preservatives, co-solvents and viscosity building agents.

Surfactants that can be used are surface-active agents that are acceptable for ophthalmic or otolaryngological uses. Useful surface active agents include but are not limited to polysorbate 80, tyloxapol, TWEEN 80 (ICI America Inc., Wilmington, Del.), PLURONIC F-68 (from BASF, Ludwigshafen, Germany) and the poloxamer surfactants can also be used. These surfactants are nonionic alkaline oxide condensates of an organic compound which contains hydroxyl groups. The concentration in which the surface active agent may be used is only limited by neutralization of the bactericidal effects on the accompanying preservatives (if present), or by concentrations which may cause irritation.

Various tonicity agents may be employed to adjust the tonicity of the composition. For example, sodium chloride, potassium chloride, magnesium chloride, calcium chloride, nonionic diols, preferably glycerol, dextrose and/or mannitol may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm).

An appropriate buffer system (e.g., sodium phosphate, sodium acetate, sodium citrate, sodium borate or boric acid) may be added to the compositions to prevent pH drift under storage conditions. The particular concentration will vary, depending on the agent employed.

Topical ophthalmic products are typically packaged in multidose form. Preservatives are thus required to prevent microbial contamination during use. Suitable preservatives include: benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% W/W. Unit dose compositions of the present invention will be sterile, but typically unpreserved. Such compositions, therefore, generally will not contain preservatives.

Co-solvents and viscosity building agents may be added to the compositions to improve the characteristics of the compositions. Such materials can include nonionic water-soluble polymer. Other compounds designed to lubricate, “wet,” approximate the consistency of endogenous tears, aid in natural tear build-up, or otherwise provide temporary relief of dry eye symptoms and conditions upon ocular administration the eye are known in the art. Such compounds may enhance the viscosity of the composition, and include, but are not limited to: monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as, polyvinyl alcohol, polyvinylpyrrolidone, povidone and carbomers, such as, carbomer 934P, carbomer 941, carbomer 940, carbomer 974P. Other compounds may also be added to the ophthalmic compositions of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

In some embodiments, the compositions disclosed herein comprise at least one suspending agent. One class of suspending agents are polymers prepared from at least about 90%, or from at least about 95%), by weight, based on the total weight of monomers present, of one or more carboxyl-containing monoethylenically unsaturated monomers. Acrylic acid is a suitable carboxyl-containing monoethylenically unsaturated monomer, but other ethylenically unsaturated, polymerizable carboxyl-containing monomers may be employed. These include: methacrylic acid, ethacrylic acid, β-methylacrylic acid (crotonic acid), cis-α-methylcrotonic acid (angelic acid), trans-α-methylcrotonic acid (tiglic acid), α-butylcrotonic acid, α-phenyl acrylic acid, α-benzylacrylic acid, α-cyclohexylacrylic acid, β-phenylacrylic acid (cinnamic acid), coumaric acid (o-hydroxycinnamic acid), coumaric acid (p-hydroxy coumaric acid), and the like, which can be used in addition to, or instead of, acrylic acid.

The carboxyl-containing polymers prepared from these monethylenically unsaturated monomers may be lightly cross-linked by employing a small percentage, i.e., from about 0.5% to about 5%, or from about 0.2% to about 3%, based on the total weight of monomers present, of a polyfunctional cross-linking agent. Such cross-linking agents including non-polyalkenyl poly ether difunctional cross-linking monomers, such as: divinyl glycol; 3,4-dihydroxy-hexa-I,5-diene; 2,5-dimethyl-I, 5-hexadiene; divinylbenzene; N,N-diallylacryiamide; N,″N-diallylmethacrylamide; and the like.

Various lightly cross-linked polymers are commercially available, or may be generally prepared by suspension or emulsion polymerization, using conventional free radical polymerization catalysts. In general, such polymers will range in molecular weight from about 250,000 to about 4,000,000, or from about 500,000 to about 2,000,000. The lightly cross-linked polymers can be made from a carboxyl-containing monomer or monomers as the sole monoethylenically unsaturated monomer present, together with the cross-linking agent or agents. They can also be polymers in which up to about 40%), or within the range of about 0% to about 20% by weight, of the carboxyl-containing monoethylenically unsaturated monomer or monomers has been replaced by one or more non-carboxyl-containing monoethylenically unsaturated monomers containing only physiologically and ophthalmologically innocuous substituents, including acrylic and methacrylic acid esters such as methyl methacrylate, ethyl acrylate, butyl acrylate, 2-ethylhexylacrylate, vinyl acetate, 2-hydroxyethylmethacrylate, 3-hydroxypropylacrylate, and the like.

Another class of lightly cross-linked polymers are carboxyl-containing polymer prepared by suspension polymerization of acrylic acid and divinyl glycol, including NOVEON AA-1 polycarbophil (available from Lubrizol). Other lightly cross-linked carboxy-containing polymers include various carbomers, such as Carbopol carbomers (available from Lubrizol). According to various aspects, the suspending agent is a carboxvinyl polymer selected from polycarbophil and carbomer.

In some embodiments, the compositions disclosed herein comprise at least one cellulose or its derivative. In some embodiments, the compositions disclosed herein comprises at least one non-ionic cellulose derivative as a supplemental suspending agent. Representative agents include hydroxypropylmethyl cellulose (“HPMC”) or hydroxypropylcellulose (“HPC”).

According to some embodiments, the compositions disclosed herein comprise one or more biocompatible polymers, such as poly (ethylene glycol), poly (lactic acid), lactic acid-glycolic acid copolymer, lactose, phosphatidylcholine, polylactide, polyglycolide, hydroxypropylcellulose, waxes, polyesters, polyanhydrides (polyanhydride), polyamide, phosphorus groups (based) polymers, poly (cyanoacrylates), polyurethanes, poly (ortho esters), poly dihydropyran, polyacetals, biodegradable polymers, polypeptide, hydrogels, and carbohydrates.

In some embodiments, a subject composition may optionally comprise ophthalmically acceptable carriers, additives, diluents, or a combination thereof. For ophthalmic application, solutions or medicaments may be prepared using a physiological saline solution as a carrier or diluent. Ophthalmic solutions may be maintained at a physiologic pH with an appropriate buffer system. The formulations may also contain conventional additives, such as pharmaceutically acceptable buffers, preservatives, stabilizers and surfactants.

Pharmaceutical compositions described herein and for use in accordance with the articles and methods described herein may include a pharmaceutically acceptable excipient, carrier, or diluent. A pharmaceutically acceptable excipient or pharmaceutically acceptable carrier or pharmaceutically acceptable diluent may include a non-toxic, inert solid, semi-solid or liquid filler, diluent, encapsulating material, or formulation auxiliary of any suitable type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives, and antioxidants can also be present in the composition, according to the judgment of the formulator. As would be appreciated by one of skill in this art, the excipients may be chosen based on the route of administration as described below, the pharmaceutical agent being delivered, time course of delivery of the agent, etc.

The pharmaceutical agent may be present in the composition in any suitable amount. In some embodiments, the pharmaceutical agent is present in an amount of at least about 0.00001%, at least about 0.0001%, at least about 0.001% w:v, at least about 0.01%, at least about 0.1%, at least about 0.5%, at least about 1%, at least about 2%, at least about 3%, at least about 4%, at least about 5%, at least about 6%, at least about 7%, at least about 8%, at least about 10%, at least about 12%, at least about 15%, at least about 20%, at least about 40%, at least about 60%, or at least about 80% of the composition in total. In some cases, the pharmaceutical agent may be present in the composition in an amount of less than or equal to about 100%, less than or equal to about 90%, less than or equal to about 80%, less than or equal to about 60%, less than or equal to about 40%, less than or equal to about 20%, less than or equal to about 15%, less than or equal to about 12%, less than or equal to about 10%, less than or equal to about 8%, less than or equal to about 7%, less than or equal to about 6%, less than or equal to about 5%, less than or equal to about 4%, less than or equal to about 3%, less than or equal to about 2%, or less than or equal to about 1% of the composition in total. Combinations of the above-referenced ranges are also possible (e.g., an amount of less than or equal to about 20% and at least about 1% of the composition), from about 0.1% to about 2%, or from about 0.25% to about 1%). In some embodiments, the pharmaceutical agent is present in the composition in a concentration of about 0.25% or abut 1% in total. In some embodiments, the pharmaceutical agent is present in the above ranges but in w:v.

A subject composition may include one or more buffers. Examples include, but are not limited to, acetate buffers, citrate buffers, phosphate buffers, borate buffers, lactate buffers, NaOH/trolamine buffers, or a combination thereof such as phosphate and citrate, or borate and citrate. Acids or bases, such as HCl and NaOH, may be used to adjust the pH of these formulations as needed. The amount of buffer used may vary. In some embodiments, the buffer may have a concentration in a range of about 1 nM to about 100 mM.

A subject composition may include one or more preservatives. The preservatives may vary, and may include any compound or substance suitable for reducing or preventing microbial contamination in an ophthalmic liquid subject to multiple uses from the same container. Preservatives that may be used in the pharmaceutical compositions disclosed herein include, but are not limited to, cationic preservatives such as quaternary ammonium compounds including benzalkonium chloride, polyquaternium-1 (Polyquad®), and the like; guanidine-based preservatives including PHMB, chlorhexidine, and the like; chlorobutanol; mercury preservatives such as thimerosal, phenylmercuric acetate and phenylmercuric nitrate; and other preservatives such as benzyl alcohol. In some embodiments, a preservative may have a concentration of about 10 ppm to about 200 ppm, about 10 ppm to about 300 ppm, or about 50 ppm to about 150 ppm.

A subject composition may include one or more surfactants of the following classes: alcohols; amine oxides; block polymers; carboxylated alcohol or alkylphenol ethoxylates; carboxylic acids/fatty acids; ethoxylated alcohols; ethoxylated alkylphenols; ethoxylated aryl phenols; ethoxylated fatty acids; ethoxylated; fatty esters or oils (animal and vegetable); fatty esters; fatty acid methyl ester ethoxylates; glycerol esters; glycol esters; lanolin-based derivatives; lecithin and lecithin derivatives; lignin and lignin derivatives; methyl esters; monoglycerides and derivatives; polyethylene glycols; polymeric surfactants; propoxylated & ethoxylated fatty acids, alcohols, or alkyl phenols; protein-based surfactants; sarcosine derivatives; sorbitan derivatives; sucrose and glucose esters and derivatives. The amount of surfactant may vary. In some embodiments, the amount of any surfactant such as those listed above may be about 0.001 to about 5%, about 0.1% to about 2%, or about 0.1% to about 1%.

A subject composition may include one or more tonicity agents (tonicity adjusters). The tonicity agents may vary, and may include any compound or substance useful for adjusting the tonicity of an ophthalmic liquid. Examples include, but are not limited to, salts, particularly sodium chloride or potassium chloride, organic compounds such as propylene glycol, mannitol, or glycerin, or any other suitable ophthalmically acceptable tonicity agent. The amount of tonicity agent may vary depending upon whether an isotonic, hypertonic, or hypotonic liquid is desired. In some embodiments, the amount of a tonicity agent such as those listed above may be at least about 0.0001% up to about 1%, about 2%, or about 5%. In some embodiments, the tonicity agent comprises glycerin and/or sodium chloride.

A tonicity agent (such as one described herein) may be present at a suitable concentration in a composition and/or formulation including the coated particles described herein. In certain embodiments, the concentration of the tonicity agent is greater than or equal to about 0.003 wt %, greater than or equal to about 0.01 wt %, greater than or equal to about 0.03 wt %, greater than or equal to about 0.1 wt %, greater than or equal to about 0.3 wt %, greater than or equal to about 1 wt %, greater than or equal to about 3 wt %, greater than or equal to about 10 wt %, greater than or equal to about 20 wt %, or greater than or equal to about 30 wt %. In certain embodiments, the concentration of the tonicity agent is less than or equal to about 30 wt %, less than or equal to about 10 wt %, less than or equal to about 3 wt %, less than or equal to about 1 wt %, less than or equal to about 0.3 wt %, less than or equal to about 0.1 wt %, less than or equal to about 0.03 wt %, less than or equal to about 0.01 wt %, or less than or equal to about 0.003 wt %. Combinations of the above-noted ranges are possible (e.g., a concentration of greater than or equal to about 0.1 wt % and less than or equal to about 10 wt %). Other ranges are also possible. In certain embodiments, the concentration of the tonicity agent is about 0.1-1 wt %. In certain embodiments, the concentration of the tonicity agent is about 0.5-3 wt %. In certain embodiments, the concentration of the tonicity agent is about 0.25 wt %. In certain embodiments, the concentration of the tonicity agent is about 0.45 wt %. In certain embodiments, the concentration of the tonicity agent is about 0.9 wt %. In certain embodiments, the concentration of the tonicity agent is about 1.2 wt %. In certain embodiments, the concentration of the tonicity agent is about 2.4 wt %. In certain embodiments, the concentration of the tonicity agent is about 5 wt %.

In some embodiments, a tonicity agent may be present in a composition and/or formulation in one or more of the above-noted ranges during a formation process and/or a dilution process described herein. In certain embodiments, a tonicity agent may be present in a composition and/or formulation in one or more of the above-noted ranges in a final product.

The osmolality of a subject composition may be hypotonic, isotonic, or hypertonic. For example, a subject composition may have an osmolarity of about 200-250 mOsm/kg, about 250-280 mOsm/kg, about 280-320 mOsm/kg, about 290-310 mOsm/kg, about 295-305 mOsm/kg, about 300 mOsm/kg (isotonic), about 300-350 mOsm/kg, or any osmolarity in a range bounded by any of these values. To achieve a formulation of an osmolarity of about 300 mOsm/kg, the concentration of sodium chloride in the formulation is typically about 0.9%. A combination of 1.2% glycerin and 0.45% sodium chloride generally also yields an isotonic solution.

A subject composition may include an antioxidant such as sodium metabisulfite, sodium thiosulfate, acetylcysteine, butylated hydroxyanisole, or butylated hydroxytoluene.

A subject composition may include a chelating agent such as edetate disodium.

A subject composition may be suitable for administration to an eye, such as topical administration to the eye or direct injection into the eye.

Generally, it is desired that a formulation is sterile before or upon administration to a subject. A sterile formulation is essentially free of pathogenic microorganisms, such as bacteria, microbes, fungi, viruses, spores, yeasts, molds, and others generally associated with infections. In some embodiments, compositions and/or formulations including the coated particles described herein may be subject to an aseptic process and/or other sterilization process. An aseptic process typically involves sterilizing the components of a formulation, final formulation, and/or container closure of a drug product through a process such as heat, gamma irradiation, ethylene oxide, or filtration and then combining in a sterile environment. In some cases, an aseptic process is preferred. In other embodiments, terminal sterilization is preferred.

Examples of other sterilization methods include radiation sterilization (e.g., gamma, electron, or x-ray radiation), heat sterilization, sterile filtration, and ethylene oxide sterilization. The terms “radiation” and “irradiation” are used herein interchangeably. Unlike other sterilization methods, radiation sterilization has the advantage of high penetrating ability and instantaneous effects, without the need to control temperature, pressure, vacuum, or humidity in some instances. In certain embodiments, the radiation used to sterilize the coated particles described herein is gamma radiation. Gamma radiation may be applied in an amount sufficient to kill most or substantially all of the microbes in or on the coated particles. The temperature of the coated particles described herein and the rate of radiation may be relatively constant during the entire gamma radiation period. Gamma irradiation may be performed at any suitable temperature (e.g., ambient temperature, about 40° C., between about 30 to about 50° C.). Unless otherwise indicated, measurements of gamma irradiation described herein refer to ones performed at about 40° C.

In embodiments in which a sterilization process is used, it may be desired that the process does not: (1) significantly change the particle size of the coated particles described herein; (2) significantly change the integrity of the active ingredient (such as a drug) of the coated particles described herein; and (3) generate unacceptable concentrations of impurities during or following the process. In certain embodiments, the impurities generated during or following the process are degradants of the active ingredient of the coated particles described herein. For example, when the active ingredient is loteprednol etabonate (LE), degradants of LE may include 11β,17α-dihydroxy-3-oxoandrosta-1,4-diene-17-carboxylic acid (PJ-90), 17α-[(ethoxycarbonyl)oxy]-11β-hydroxy-3-oxoandrosta-1,4-diene-17β-carboxylic acid (PJ-91), 17α-[(ethoxycarbonyl)oxy]-11β-hydroxy-3-oxoandrosta-4-ene-17-carboxylic acid chloromethyl ester (tetradeca), and/or 17α-[(ethoxycarbonyl)oxy]-3,11-dioxoandrosta-1,4-diene-17-carboxylic acid chloromethyl ester (11-keto).

In certain embodiments, a process used to sterilize a composition and/or formulation described herein results in the presence of one or more degradants in the formulation at less than or equal to about 10 wt % (relative to the weight of the undegraded drug), less than or equal to about 3 wt %, less than or equal to about 2 wt %, less than or equal to about 1.5 wt %, less than or equal to about 1 wt %, less than or equal to about 0.9 wt %, less than or equal to about 0.8 wt %, less than or equal to about 0.7 wt %, less than or equal to about 0.6 wt, less than or equal to about 0.5 wt %, less than or equal to about 0.4 wt %, less than or equal to about 0.3 wt %, less than or equal to about 0.2 wt %, less than or equal to about 0.15 wt %, less than or equal to about 0.1 wt %, less than or equal to about 0.03 wt %, less than or equal to about 0.01 wt %, less than or equal to about 0.003 wt %, or less than or equal to about 0.001 wt %. In some embodiments, the process results in a degradant in the formulation at greater than or equal to about 0.001 wt %, greater than or equal to about 0.003 wt %, greater than or equal to about 0.01 wt %, greater than or equal to about 0.03 wt %, greater than or equal to about 0.1 wt %, greater than or equal to about 0.3 wt %, greater than or equal to about 1 wt %, greater than or equal to about 3 wt %, or greater than or equal to about 10 wt %. Combinations of the above-referenced ranges are also possible (e.g., less than or equal to about 1 wt % and greater than or equal to about 0.01 wt %). Other ranges are also possible.

In some embodiments, a composition and/or formulation subjected to gamma irradiation includes a degradant having a concentration at one or more of the above-noted ranges. In one set of embodiments, the drug is loteprednol etabonate and the degradant is PJ-90, PJ-91, tetradeca, and/or 11-keto. In certain embodiments, one or more, or each, of the degradants is present in a composition and/or formulation at one or more of the above-noted ranges (e.g., less than or equal to about 1 wt %, less than or equal to about 0.9 wt %, less than or equal to about 0.8 wt %, less than or equal to about 0.7 wt %, less than or equal to about 0.6 wt %, less than or equal to about 0.5 wt %, less than or equal to about 0.4 wt %, less than or equal to about 0.3 wt %, less than or equal to about 0.2 wt %, or less than or equal to about 0.1 wt %). Other ranges are also possible.

In some embodiments, one or more additives are included in the composition and/or formulation to help achieve a relatively low amount of one or more degradants. For example, the presence of glycerin in a loteprednol etabonate formulation resulted in relatively low amounts of the degradant tetradeca after the formulation was sterilized with gamma irradiation, compared to a loteprednol etabonate formulation that did not include glycerin.

Some exemplary compositions that can be used with the loteprednol etabonate forms I and II as disclosed here include those described in WO2016/123079 and US 2016/0213609, and US 2005/0182039, and in Coffey et al. Clinical Ophthalmology 7:299-312 (2013), the entire relevant teachings of all of which are incorporated herein by reference.

According to various aspects, loteprednol etabonate crystalline forms I and II may be present as microparticles and/or nanoparticles. Typically, microparticles have a size greater than about 1,000 nm and less than about 1,000 micrometer (micron). Typically, “nanoparticles” has a size greater than about 1 nm and less than about 1,000 nm, such as greater than about 1 nm and less than about 1,000 nm.

In some embodiments, the particles of loteprednol etabonate crystalline forms I and II as described herein have Dᵥ₉₀ less than about 5 micron, Dᵥ₉₀ less than about 3 micron, Dᵥ₉₀ less than about 1,000 nm (or 1 micron).

In some embodiments, the particles of loteprednol etabonate crystalline forms I and II as described herein have Dᵥ₅₀ less than about 5 micron, Dᵥ₅₀ less than about 3 micron, Dᵥ₅₀ less than about 1,000 nm (or 1 micron), Dᵥ₅₀ less than about 700 nm, or Dᵥ₅₀ less than about 500 nm.

A subject composition may be administered by any suitable route, such as orally in any acceptable form (e.g., tablet, liquid, capsule, powder, and the like); topically in any acceptable form (e.g., patch, eye drops, creams, gels, nebulization, punctal plug, drug eluting contact, iontophoresis, and ointments); by injection in any acceptable form (e.g., periocular, intravenous, intraperitoneal, intramuscular, subcutaneous, parenteral, and epidural); by inhalation; and by implant or the use of reservoirs (e.g., subcutaneous pump, intrathecal pump, suppository, biodegradable delivery system, non-biodegradable delivery system and other implanted extended or slow release device or formulation). The target may be the eye or another organ or tissue. In some embodiments, a subject composition is administered to an eye in order to deliver the pharmaceutical agent to a tissue in the eye of the subject.

A subject composition may be administered at any suitable frequency. For example, two or more doses of a subject composition may be administered to subject, e.g. to an eye of a subject, wherein the period between consecutive doses is at least about 4 hours, at least about 6 hours, at least about 8 hours, at least about 12 hours, at least about 24 hours, at least about 36 hours, or at least about 48 hours, at least a week, or at least a month.

A subject composition may be administered to treat, diagnose, prevent, or manage a disease or condition in a subject, including a human being or a non-human animal, such as a mammal. In some embodiments, the condition is an ocular condition, such as condition affecting the anterior or front of the eye, such as post-surgical inflammation, uveitis, infections, aphakia, pseudophakia, astigmatism, blepharospasm, cataract, conjunctival diseases, conjunctivitis, corneal diseases, corneal ulcer, dry eye syndromes, eyelid diseases, lacrimal apparatus diseases, lacrimal duct obstruction, myopia, presbyopia; pupil disorders, corneal neovascularization; refractive disorders, and strabismus. Glaucoma can be considered to be a front of the eye ocular condition in some embodiments because a clinical goal of glaucoma treatment can be to reduce a hypertension of aqueous fluid in the anterior chamber of the eye (i.e., reduce intraocular pressure).

The leading causes of vision impairment and blindness are conditions linked to the posterior segment of the eye. These conditions may include, without limitation, age-related ocular degenerative diseases such as, macular degeneration, including acute macular degeneration, exudative and non-exudative age related macular degeneration (collectively AMD), proliferative vitreoretinopathy (PVR), retinal ocular condition, retinal damage, macular edema (e.g., cystoid macular edema (CME) or (diabetic macular edema (DME)), endophthalmitis; intraocular melanoma; acute macular neuroretinopathy; Behcet's disease; choroidal neovascularization; uveitis; diabetic uveitis; histoplasmosis; infections, such as fungal or viral-caused infections; edema; multifocal choroiditis; ocular trauma which affects a posterior ocular site or location; ocular tumors; retinal disorders, such as central retinal vein occlusion, diabetic retinopathy (including proliferative diabetic retinopathy), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, retinoblastoma. Glaucoma can be considered a posterior ocular condition in some embodiments because the therapeutic goal is to prevent the loss of or reduce the occurrence of loss of vision due to damage to or loss of retinal cells or optic nerve cells (i.e., neuroprotection). In fact, certain forms of glaucoma are not characterized by high 10P, but mainly by retinal degeneration alone.

Some embodiments include administering a composition disclosed herein to treat inflammation, macular degeneration, macular edema, uveitis, dry eye, or glaucoma.

### Preparation of Coated Particles

While there are many potential ways to coat drug or core particles with a surface-altering agent, typically this could involve milling the particles (such as drug particles) with a surface-altering agent or incubating particles in an aqueous solution in the presence of a surface-altering agent. Another useful method involves dissolving a drug in an organic solvent and emulsifying the solution in water using the surface-altering agent as a surfactant, then removing the organic solvent by evaporation (e.g. by rotary evaporation). Combinations of these methods may also be used.

In a wet milling process, milling can be performed in a dispersion (e.g., an aqueous dispersion) containing one or more surface-altering agents, a grinding medium, a solid to be milled (e.g., a solid pharmaceutical agent), and a solvent. Any suitable amount of a surface-altering agent can be included in the solvent. In some embodiments, a surface-altering agent may be present in the solvent in an amount of at least about 0.001% (wt % or % weight to volume (w:v)), at least about 0.01%, at least about 0.1%, at least about 0.5%, at least about 1%, at least about 2%, at least about 3%, at least about 4%, at least about 5%, at least about 6%, at least about 7%, at least about 8%, at least about 10%, at least about 12%, at least about 15%, at least about 20%, at least about 40%, at least about 60%, or at least about 80% of the solvent. In some cases, the surface-altering agent may be present in the solvent in an amount of about 100% (e.g., in an instance where the surface-altering agent is the solvent). In other embodiments, the surface-altering agent may be present in the solvent in an amount of less than or equal to about 100%, less than or equal to about 80%, less than or equal to about 60%, less than or equal to about 40%, less than or equal to about 20%, less than or equal to about 15%, less than or equal to about 12%, less than or equal to about 10%, less than or equal to about 8%, less than or equal to about 7%, less than or equal to about 6%, less than or equal to about 5%, less than or equal to about 4%, less than or equal to about 3%, less than or equal to about 2%, or less than or equal to about 1% of the solvent. Combinations of the above-referenced ranges are also possible (e.g., an amount of less than or equal to about 5% and at least about 1% of the solvent). Other ranges are also possible. In certain embodiments, the surface-altering agent is present in the solvent in an amount of about 0.01-2% of the solvent. In certain embodiments, the surface-altering agent is present in the solvent in an amount of about 0.2-20% of the solvent. In certain embodiments, the surface-altering agent is present in the solvent in an amount of about 0.1% of the solvent. In certain embodiments, the surface-altering agent is present in the solvent in an amount of about 0.4% of the solvent. In certain embodiments, the surface-altering agent is present in the solvent in an amount of about 1% of the solvent. In certain embodiments, the surface-altering agent is present in the solvent in an amount of about 2% of the solvent. In certain embodiments, the surface-altering agent is present in the solvent in an amount of about 5% of the solvent. In certain embodiments, the surface-altering agent is present in the solvent in an amount of about 10% of the solvent.

The particular range chosen may influence factors that may affect the ability of the particles to penetrate mucus such as the stability of the coating of the surface-altering agent on the particle surface, the average thickness of the coating of the surface-altering agent on the particles, the orientation of the surface-altering agent on the particles, the density of the surface altering agent on the particles, surface-altering agent:drug ratio, drug concentration, the size, dispersibility, and polydispersity of the particles formed, and the morphology of the particles formed.

The pharmaceutical agent may be present in the solvent in any suitable amount. In some embodiments, the pharmaceutical agent is present in an amount of at least about 0.00001%, at least about 0.0001%, at least about 0.001% w:v, at least about 0.01%, at least about 0.1%, at least about 0.5%, at least about 1%, at least about 2%, at least about 3%, at least about 4%, at least about 5%, at least about 6%, at least about 7%, at least about 8%, at least about 10%, at least about 12%, at least about 15%, at least about 20%, at least about 40%, at least about 60%, or at least about 80% of the solvent. In some cases, the pharmaceutical agent may be present in the solvent in an amount of less than or equal to about 100%, less than or equal to about 90%, less than or equal to about 80%, less than or equal to about 60%, less than or equal to about 40%, less than or equal to about 20%, less than or equal to about 15%, less than or equal to about 12%, less than or equal to about 10%, less than or equal to about 8%, less than or equal to about 7%, less than or equal to about 6%, less than or equal to about 5%, less than or equal to about 4%, less than or equal to about 3%, less than or equal to about 2%, or less than or equal to about % of the solvent. Combinations of the above-referenced ranges are also possible (e.g., an amount of less than or equal to about 20% and at least about 1% of the solvent). In some embodiments, the pharmaceutical agent is present in the above ranges but in w:v

The ratio of surface-altering agent to pharmaceutical agent in a solvent may also vary. In some embodiments, the ratio of surface-altering agent to pharmaceutical agent may be at least 0.001:1 (weight ratio, molar ratio, or w:v ratio), at least 0.01:1, at least 0.01:1, at least 1:1, at least 2:1, at least 3:1, at least 5:1, at least 10:1, at least 25:1, at least 50:1, at least 100:1, or at least 500:1. In some cases, the ratio of surface-altering agent to pharmaceutical agent may be less than or equal to 1000:1 (weight ratio or molar ratio), less than or equal to 500:1, less than or equal to 100:1, less than or equal to 75:1, less than or equal to 50:1, less than or equal to 25:1, less than or equal to 10:1, less than or equal to 5:1, less than or equal to 3:1, less than or equal to 2:1, less than or equal to 1:1, or less than or equal to 0.1:1. Combinations of the above-referenced ranges are possible (e.g., a ratio of at least 5:1 and less than or equal to 50:1). Other ranges are also possible.

It should be appreciated that while in some embodiments the stabilizer used for milling forms a coating on a particle surface, which coating renders particle mucus penetrating, in other embodiments, the stabilizer may be exchanged with one or more other surface-altering agents after the particle has been formed. For example, in one set of methods, a first stabilizer/surface-altering agent may be used during a milling process and may coat a surface of a core particle, and then all or portions of the first stabilizer/surface-altering agent may be exchanged with a second stabilizer/surface-altering agent to coat all or portions of the core particle surface. In some cases, the second stabilizer/surface-altering agent may render the particle mucus penetrating more than the first stabilizer/surface-altering agent. In some embodiments, a core particle having a coating including multiple surface-altering agents may be formed.

Any suitable grinding medium can be used for milling. In some embodiments, a ceramic and/or polymeric material and/or a metal can be used. Examples of suitable materials may include zirconium oxide, silicon carbide, silicon oxide, silicon nitride, zirconium silicate, yttrium oxide, glass, alumina, alpha-alumina, aluminum oxide, polystyrene, poly(methyl methacrylate), titanium, steel. A grinding medium may have any suitable size. For example, the grinding medium may have an average diameter of at least about 0.1 mm, at least about 0.2 mm, at least about 0.5 mm, at least about 0.8 mm, at least about 1 mm, at least about 2 mm, or at least about 5 mm. In some cases, the grinding medium may have an average diameter of less than or equal to about 5 mm, less than or equal to about 2 mm, less than or equal to about 1 mm, less than or equal to about 0.8, less than or equal to about 0.5 mm, or less than or equal to about 0.2 mm. Combinations of the above-referenced ranges are also possible (e.g., an average diameter of at least about 0.5 millimeters and less than or equal to about 1 mm). Other ranges are also possible.

Any suitable solvent may be used for milling. The choice of solvent may depend on factors such as the solid material (e.g., pharmaceutical agent) being milled, the particular type of stabilizer/surface-altering agent being used (e.g., one that may render the particle mucus penetrating), the grinding material be used, among other factors. Suitable solvents may be ones that do not substantially dissolve the solid material or the grinding material, but dissolve the stabilizer/surface-altering agent to a suitable degree. Non-limiting examples of solvents may include water, buffered solutions, other aqueous solutions, alcohols (e.g., ethanol, methanol, butanol), and mixtures thereof that may optionally include other components such as pharmaceutical excipients, polymers, pharmaceutical agents, salts, preservative agents, viscosity modifiers, tonicity modifier, taste masking agents, antioxidants, pH modifier, and other pharmaceutical excipients. In other embodiments, an organic solvent can be used.

After milling, a dilution process may be used to form and/or modify coated particles from a suspension. The coated particles may comprise a core material, one or more surface-altering agents, and other components, such as solvents, tonicity agents, chelating agents, salts anti-microbial agents, and buffers (e.g., a sodium citrate and citric acid buffer). A dilution process may be used to achieve a target dosing concentration by diluting a solution or suspension of particles that were coated during a milling step, with or without the additional of surface-altering agents and/or other components. In certain embodiments, a dilution process may be used to exchange a first surface-altering agent with a second surface-altering agent from a surface of a particle as described herein.

The dilution process may be performed using a product vessel or any other suitable apparatus. In certain embodiments, the suspension is diluted, i.e., mixed or otherwise processed with a diluent, in the product vessel. The diluent may contain solvents, surface-altering agents, tonicity agents, chelating agents, salts, or anti-microbial agents, or a combination thereof, as described herein. The suspension and the diluent may be added into the product vessel at the same time or different times. In certain embodiments when the suspension is obtained from a milling process involving milling media, the milling media may be separated from the suspension before the suspension is added into the product vessel. The suspension, the diluent, or the mixture of the suspension and the diluent may be stirred and/or shaken, or otherwise agitated, to form the coated particles described herein. The temperature and/or pressure of the suspension, the diluent, or the mixture may also be individually increased or decreased to form the coated particles. In some embodiments, the suspension and the diluent are processed in the product vessel under an inert atmosphere (e.g., nitrogen or argon) and/or protected from light.

In some embodiments, the loteprednol etabonate form I or form II as disclosed herein are present in the subject compositions disclosed herein at about 0.1% to 2% (such as about 0.1%-about 1.5%; about 0.2%-about 1%; about 0.25%; about 0.5%; about 0.38%; and about 1%) by weight based on the compositions. In some embodiments, the loteprednol etabonate form I or form II as disclosed herein are present in the subject compositions disclosed herein at about 0.1% w/v to 2% w/v (such as about 0.1% w/v-about 1.5% w/v; about 0.2% w/v-about 1% w/v; about 0.25% w/v; about 0.5% w/v; about 0.38% w/v; and about 1% w/v).

### Methods

Provided herein are methods of treating an ocular condition in a subject in need thereof. Also provided herein are methods of sustaining an ophthalmically efficacious level of loteprednol etabonate in a first eye tissue in a subject in need thereof.

Thus, in one aspect, provided herein are methods of treating an ocular condition in a subject in need thereof, comprising administering a pharmaceutical composition provided herein to an eye tissue of the subject.

In some embodiments, the ocular condition is inflammation, macular degeneration, macular edema, uveitis, glaucoma, or dry eye.

In some embodiments, the ocular condition is dry eye.

In some embodiments, the ocular condition is post-surgical inflammation.

In another aspect, provided herein are methods of sustaining an ophthalmically efficacious level of loteprednol etabonate in a first eye tissue in a subject in need thereof, comprising administering a pharmaceutical composition provided herein to a second eye tissue of the subject, wherein the level of loteprednol etabonate is sustained for at least 12 hours after administration.

In some embodiments, the level of loteprednol etabonate is sustained for 12 hours, 14 hours, 16 hours, 18 hours, 20 hours, 22 hours, 24 hours, 30 hours, 36 hours, 40 hours, or 48 hours after administration.

In some embodiments, the first eye tissue is a palpebral conjunctiva, a fornix conjunctiva, a bulbar conjunctiva, or a cornea.

In some embodiments of these methods, the pharmaceutical composition is administered to a tissue in the front of the eye of the subject.

In some embodiments, the pharmaceutical composition is administered to a tissue in the back of the eye of the subject.

In some embodiments, the first eye tissue is a retina, a macula, a sclera, a cornea, a lid, an aqueous humor, or a choroid.

In some embodiments, provided herein are methods of treating dry eye or post-surgical inflammation in a subject in need thereof, comprising administering a pharmaceutical composition disclosed herein to an eye tissue of the subject.

In some embodiments, provided herein are methods of sustaining an ophthalmically efficacious level of loteprednol etabonate in a first eye tissue in a subject in need thereof, comprising administering a pharmaceutical composition disclosed herein to a second eye tissue of the subject, wherein the level of loteprednol etabonate is sustained for at least 12 hours after administration.

In some embodiments, one or more additional therapeutic agents, or other agents, can be used in combination with the agent in the methods disclosed herein. The one or more additional therapeutic agents can be administered to a patient simultaneously or sequentially.

In some embodiments, the additional therapeutic agent is an anti-angiogenic agent, cholinergic agonist, TRP-1 receptor modulator, a calcium channel blocker, a mucin secretagogue, MUC1 stimulant, a calcineurin inhibitor, a corticosteroid, a P2Y2 receptor agonist, a muscarinic receptor agonist, another JAK inhibitor, Bcr-Abl kinase inhibitor, Flt-3 kinase inhibitor, RAF kinase inhibitor, and FAK kinase. In some embodiments, the additional therapeutic agent is a tetracycline derivative (e.g., minocycline or doxycline).

In some embodiments, the additional therapeutic agent(s) are demulcent eye drops (also known as “artificial tears”), which include, but are not limited to, compositions containing polyvinylalcohol, hydroxypropyl methylcellulose, glycerin, polyethylene glycol (e.g. PEG400), or carboxymethyl cellulose. Artificial tears can help in the treatment dry eye by compensating for reduced moistening and lubricating capacity of the tear film. In some embodiments, the additional therapeutic agent is a mucolytic drug, such as N-acetyl-cysteine, which can interact with the mucoproteins and, therefore, to decrease the viscosity of the tear film.

In some embodiments, the additional therapeutic agent includes an antibiotic, antiviral, antifungal, anesthetic, anti-inflammatory agents including steroidal and non-steroidal antiinflammatories, and anti-allergic agents. Examples of suitable medicaments include aminoglycosides such as amikacin, gentamycin, tobramycin, streptomycin, netilmycin, and kanamycin; fluoroquinolones such as ciprofloxacin, norfloxacin, ofloxacin, trovafloxacin, lomefloxacin, levofloxacin, and enoxacin; naphthyridine; sulfonamides; polymyxin; chloramphenicol; neomycin; paramomomycin; colistimethate; bacitracin; vancomycin; tetracyclines; rifampin and its derivatives (“rifampins”); cycloserine; beta-lactams; cephalosporins; amphotericins; fluconazole; flucytosine; natamycin; miconazole; ketoconazole; corticosteroids; diclofenac; flurbiprofen; ketorolac; suprofen; comolyn; lodoxamide; levocabastin; naphazoling; antazoline; pheniramimane; or azalide antibiotic.

The following embodiments are also contemplated:

### Embodiment 1

A pharmaceutical composition suitable for administration to an eye, comprising: a plurality of mucus-penetrating coated particles, each coated particle comprising a core particle comprising loteprednol etabonate crystalline form I or II, and a mucus penetration-enhancing coating comprising a surface-altering agent surrounding the core particle, wherein the surface-altering agent comprises one or more of the following components: a) a triblock copolymer comprising a hydrophilic block—hydrophobic block—hydrophilic block configuration, wherein the hydrophobic block has a molecular weight of at least about 2 kDa, and the hydrophilic blocks constitute at least about 15 wt % of the triblock copolymer, wherein the hydrophobic block associates with the surface of the core particle, and wherein the hydrophilic block is present at the surface of the coated particle and renders the coated particle hydrophilic, b) a synthetic polymer having pendant hydroxyl groups on the backbone of the polymer, the polymer having a molecular weight of at least about 1 kDa and less than or equal to about 1000 kDa, wherein the hydrolysis degree of the polymer is at least about 30% and less than about 95%, or c) a polysorbate, and at least one ophthalmically acceptable carrier, additive, or diluent, wherein the surface altering agent is present on the outer surface of the core particle at a density of at least 0.01 molecules/nm², wherein the surface altering agent is present in the pharmaceutical composition in an amount of between about 0.001% to about 5% by weight in total.

### Embodiment 2

A pharmaceutical composition suitable for treating an ocular disorder by administration to an eye, comprising: a plurality of mucus-penetrating coated particles, each coated particle comprising: a core particle comprising loteprednol etabonate crystalline form I or II, and a mucus penetration-enhancing coating comprising a surface-altering agent surrounding the core particle, wherein the surface-altering agent comprises one or more of the following components: a) a triblock copolymer comprising a hydrophilic block—hydrophobic block—hydrophilic block configuration, wherein the hydrophobic block has a molecular weight of at least about 2 kDa, and the hydrophilic blocks constitute at least about 15 wt % of the triblock copolymer, b) a synthetic polymer having pendant hydroxyl groups on the backbone of the polymer, the polymer having a molecular weight of at least about 1 kDa and less than or equal to about 1000 kDa, wherein the hydrolysis degree of the polymer is at least about 30% and less than about 95%, or c) a polysorbate, and at least one ophthalmically acceptable carrier, additive, or diluent; wherein the plurality of coated particles have an average smallest cross-sectional dimension of less than about 1 micron; and wherein the coating on the core particle is present in a sufficient amount to increase the concentration of the loteprednol etabonate in a cornea or an aqueous humor after administration when administered to the eye, compared to the concentration of the loteprednol etabonate in the cornea or the aqueous humor when administered as a core particle without the coating.

### Embodiment 3

The pharmaceutical composition of embodiments 1 or 2, wherein loteprednol etabonate crystalline form I has XRPD peaks at about 5.6, 7.7, 11.9, 14.1, 17.0 and 18.8±0.2° 2θ, and wherein loteprednol etabonate crystalline form II has XRPD peaks at about 15.0°, 18.1°, and 19.8°±0.2° 2θ.

### Embodiment 4

The pharmaceutical composition of embodiment 3, wherein the loteprednol etabonate crystalline form I has additional XRPD peaks at about 16.0, 21.0 and 22.0±0.2° 2θ, wherein the loteprednol etabonate crystalline form II has additional XRPD peaks at about 9.8°, 15.6°, 16.6°, 17.2°, 23.0°, 24.8°, and 26.3°±0.2° 2θ:

### Embodiment 5

The pharmaceutical composition of any one of embodiments 1-4, wherein the surface-altering agent is covalently attached to the core particles.

### Embodiment 6

The pharmaceutical composition of any one of embodiments 1-4, wherein the surface-altering agent is non-covalently adsorbed to the core particles.

### Embodiment 7

The pharmaceutical composition of any one of embodiments 1-6, wherein the surface-altering agent is present on the surfaces of the coated particles at a density of at least about 0.1 molecules per nanometer squared.

### Embodiment 8

The pharmaceutical composition of any one of embodiments 1-7, wherein the surface-altering agent comprises a triblock copolymer.

### Embodiment 9

The pharmaceutical composition of embodiment 9, wherein the hydrophilic blocks of the triblock copolymer constitute at least about 30 wt % of the triblock polymer and less than or equal to about 80 wt % of the triblock copolymer.

### Embodiment 10

The pharmaceutical composition of embodiment 8 or 9, wherein the hydrophobic block portion of the triblock copolymer has a molecular weight of about 3 kDa to about 8 kDa.

### Embodiment 11

The pharmaceutical composition of any one of embodiments 8-10, wherein the triblock copolymer is poly(ethylene oxide)-poly(propylene oxide)-poly(ethylene oxide).

### Embodiment 12

The pharmaceutical composition of any one of embodiments 1-11, wherein the surface-altering agent has a molecular weight of at least about 4 kDa.

### Embodiment 13

The pharmaceutical composition of any one of embodiments 1-7, wherein the surface-altering agent comprises a linear polymer having pendant hydroxyl groups on the backbone of the polymer.

### Embodiment 14

The pharmaceutical composition of any one of embodiments 1-7 and 13, wherein the surface altering agent is polyvinyl alcohol.

### Embodiment 15

The pharmaceutical composition of embodiment 14, wherein the polyvinyl alcohol has a hydrolysis degree of about 30% to about 95%.

### Embodiment 16

The pharmaceutical composition of any one of embodiments 1-15, wherein the core particles comprise loteprednol etabonate crystalline form I that is encapsulated in a polymer, a lipid, a protein, or a combination thereof.

### Embodiment 17

The pharmaceutical composition of any one of embodiments 1-16, wherein the loteprednol etabonate crystalline form I constitutes at least about 80 wt % of the core particle.

### Embodiment 18

The pharmaceutical composition of embodiment 17, wherein the loteprednol etabonate crystalline form I constitutes at least about 90 wt % of the core particle.

### Embodiment 19

The pharmaceutical composition of any one of embodiments 1-18, wherein the coated particles have an average size of about 10 nm to about 1 μm.

### Embodiment 20

The pharmaceutical composition of any one of embodiments 1-19, comprising one or more degradants of the loteprednol etabonate, and wherein the concentration of each degradant is 0.2 wt % or less relative to the weight of the loteprednol etabonate.

### Embodiment 21

The pharmaceutical composition of any one of embodiments 1-20, wherein the polydispersity index of the composition is less than or equal to about 0.5.

### Embodiment 22

The pharmaceutical composition of any one of embodiments 1-21, wherein the pharmaceutical composition is suitable for topical administration to the eye.

### Embodiment 23

The pharmaceutical composition of any one of embodiments 1-22, wherein the pharmaceutical composition is suitable for direct injection into the eye.

### Embodiment 24

The pharmaceutical composition of any one of embodiments 1-23, wherein the ophthalmically acceptable carrier, additive, or diluent comprises glycerin.

### Embodiment 25

The pharmaceutical composition of any one of embodiments 1-24, wherein the composition further comprises an ionic tonicity agent.

### Embodiment 26

The pharmaceutical composition of any one of embodiments 1-25, wherein the ionic tonicity agent is sodium chloride.

### Embodiment 27

The pharmaceutical composition of any one of embodiments 1-26, wherein the composition comprises about 0.1 to about 1% w/v sodium chloride.

### Embodiment 28

A pharmaceutical composition, comprising particles of loteprednol etabonate crystalline form I or form II; and at least one pharmaceutically acceptable carrier, additive, or diluent.

### Embodiment 29

The pharmaceutical composition of embodiment 28, wherein the particles of loteprednol etabonate are micronized or nanoparticularized.

### Embodiment 30

The pharmaceutical composition of embodiment 28, wherein the particles of loteprednol etabonate are microparticles.

### Embodiment 31

The pharmaceutical composition of embodiment 28, wherein the particles of loteprednol etabonate are nanoparticles.

### Embodiment 32

The pharmaceutical composition of embodiment 28, wherein the particles of loteprednol etabonate are a mixture of microparticles and nanoparticles.

### Embodiment 33

The pharmaceutical composition of embodiment 28, further comprising a suspending agent and/or gelling agent.

### Embodiment 34

The pharmaceutical composition of embodiment 28, further comprising a suspending agent and a cellulose or a cellulose derivative.

### Embodiment 35

The pharmaceutical composition of any one of embodiments 28-32, further comprising a suspending agent and a non-ionic cellulose derivative.

### Embodiment 36

The pharmaceutical composition of any one of embodiments 28-32, further comprising a carboxyvinyl polymer and a non-ionic cellulose derivative.

### Embodiment 37

The pharmaceutical composition of embodiment 34, wherein the particles of loteprednol etabonate have Dᵥ₉₀ is less than about 1-10 micron, or less than about 5 micron, or less than about 1 micron.

### Embodiment 38

The pharmaceutical composition of any one of embodiments 31-35, wherein the suspending agent comprises a carboxyvinyl polymer.

### Embodiment 39

The pharmaceutical composition of embodiment 36, wherein the carboxyvinyl polymer is selected from the group consisting of polycarbophil and carbomer.

### Embodiment 40

The pharmaceutical composition of any one of embodiments 33-35, wherein the non-ionic cellulose derivative is hydroxypropylmethyl cellulose.

### Embodiment 41

The pharmaceutical composition of any one of embodiments 28-32, further comprising a hydroxypropylmethyl cellulose and a carboxyvinyl polymer selected from polycarbophil or carbomer.

### Embodiment 42

A method of treating, diagnosing, preventing, or managing an ocular condition in a subject, the method comprising: administering a pharmaceutical composition of any one of embodiments 1-41 to an eye of a subject and thereby delivering the loteprednol etabonate to a tissue in the eye of the subject.

### Embodiment 43

The method of embodiment 42, wherein after administering the pharmaceutical composition topically to the eye, an ophthalmically efficacious level of the loteprednol etabonate, is delivered to a palpebral conjunctiva, a bulbar conjunctiva, a fornix conjunctiva, an aqueous humor, an anterior sclera, or a cornea for at least 12 hours after administration.

### Embodiment 44

The method of embodiment 43, wherein the loteprednol etabonate is delivered to a tissue in the front of the eye of the subject.

### Embodiment 45

The method of embodiment 43, wherein the loteprednol etabonate is delivered to a tissue in the back of the eye of the subject.

### Embodiment 46

The method of any one of embodiments 42-45, wherein the tissue is a retina, a macula, a posterior sclera, vitreous humor, or a choroid.

### Embodiment 47

The method of embodiment 42, wherein the ocular condition is inflammation, pain, macular degeneration, macular edema, uveitis, or dry eye.

### Embodiment 48

A particle, comprising a core having an exterior surface, and a mucus penetration-enhancing coating disposed on the exterior surface of the core, wherein: the core comprises a solid form of loteprednol etabonate having an XRPD pattern substantially as shown in FIG. 2 or FIG. 3; the mucus penetration-enhancing coating comprises poloxamer 407; and the ratio of solid form of loteprednol etabonate to poloxamer 407 is 2:1.

### Embodiment 49

A pharmaceutical composition, comprising the particle, or a plurality thereof, of embodiment 48, and a pharmaceutically acceptable carrier, additive, or diluent, wherein: the particle is suitable for administration to an eye; and the mucus penetration-enhancing coating is mucus penetration-enhancing.

### Embodiment 50

The pharmaceutical composition of embodiment 49, wherein poloxamer 407 is between about 0.001% to about 5% of the pharmaceutical composition by weight in total.

### Embodiment 51

A method of treating an ocular condition in a subject in need thereof, comprising administering a pharmaceutical composition of embodiment 49 to an eye tissue of the subject.

### Embodiment 52

The method of embodiment 51, wherein the ocular condition is inflammation, pain, macular degeneration, macular edema, uveitis, or dry eye.

### Embodiment 53

The method of embodiment 51, wherein the ocular condition is dry eye.

### Embodiment 54

The method of embodiment 51, wherein the ocular condition is inflammation.

### Embodiment 55

A method of sustaining an ophthalmically efficacious level of loteprednol etabonate in a first eye tissue in a subject in need thereof, comprising administering a pharmaceutical composition of embodiment 49 to a second eye tissue of the subject, wherein the level of loteprednol etabonate is sustained in the first eye tissue for at least 12 hours after administration.

### Embodiment 56

The method of embodiment 55, wherein the first eye tissue is a palpebral conjunctiva, a fornix conjunctiva, a bulbar conjunctiva, or a cornea.

### Embodiment 57

The method of embodiment 53 or embodiment 43, wherein the pharmaceutical composition is administered to a tissue in the front of the eye of the subject.

### Embodiment 58

The method of embodiment 51 or claim 55, wherein the pharmaceutical composition is administered to a tissue in the back of the eye of the subject.

### Embodiment 59

The method of embodiment 55, wherein the first eye tissue is a retina, a macula, a sclera, a cornea, a lid, an aqueous humor, or a choroid.

### EXAMPLES

### Example 1A. Synthesis of Loteprednol Etabonate Crystalline Form I

Preparation of Single Crystal Form I Seed Crystal

Five grams of loteprednol etabonate was taken and added into a mixed solution of 100 ml of ethanol, 20 ml of water, and 40 ml of acetonitrile. The mixture was heated to 50° C., hot filtered to filter off insoluble substances, and cooled to 30° C. (if a crystal was precipitated, the supernatant was taken). Then the seed crystal as prepared according to CN106279325 was added. The resulting mixture was stirred for 30 min while kept at the temperature, to precipitate a substantial amount of crystal, cooled to 0° C.-5° C., filtered, and dried. The dried crystal was determined for the water content by the Karl Fischer method, and determined to be a loteprednol etabonate anhydrate. The obtained crystal was determined, by X-ray powder diffraction (XRPD), to have characteristic peaks at positions of 2 theta=5.6°, 7.7°, 11.9°, 14.1°, 16.0°, 17.0°, 18.8°, 21.0°, and 22.0°, as shown in FIG. 2.

### Example 1B. Synthesis of Loteprednol Etabonate Crystal Form II

Preparation of Single Crystal Form II Seed Crystal

Five grams of loteprednol etabonate was taken and added into a mixed solution of 100 ml of ethanol, 20 ml of water, and 20 ml of acetonitrile. The mixture was heated to 50° C., hot filtered to filter off insoluble substances, cooled to 30° C. (if a crystal was precipitated, the supernatant was taken). Then the seed crystal as prepared according to CN106279324 was added therein. The resulting mixture was stirred for 30 min while kept at the temperature, to precipitate a substantial amount of crystal, cooled to 0° C.-5° C., filtered, and dried. The dried crystal was analyzed by TG-DTA, with a loss in weight of about 3.7%, and determined to be a loteprednol etabonate monohydrate. The obtained crystal was determined, by X-ray powder diffraction (XRPD), to have characteristic peaks at positions of 2 theta=9.8°, 15.0°, 15.6°, 16.6°, 17.2°, 18.1°, 19.8°, 23.0°, 24.8°, and 26.3° as shown in FIG. 3.

### Example 2: Crystalline Form I or II of Loteprednol Etabonate Formulated as Mucus Penetrating Particles (MPP)

Crystalline form I and form II of loteprednol etabonate are formulated as mucus penetrating particles (MPP). Specifically, crystalline forms I and II from Example 1A and 1B are milled in the presence of PLURONIC® F127 (F127) to determine whether F127 1) aids particle size reduction to several hundreds of nanometers and 2) physically (non-covalently) coats the surface of generated nanoparticles with a mucoinert coating that would minimize particle interactions with mucus constituents and prevent mucus adhesion.

A milling procedure is employed in which an aqueous dispersion containing coarse drug particles and PLURONIC® F127 (F127) is milled with grinding medium until particle size and polydispersity index (a measure of the width of the particle size distribution) are reduced to less than 1 μm (z-averaged, as measured by dynamic light scattering) and approximately less than or equal to 0.5, respectively. In this example, suspensions are buffered using DPBS (Dulbecco's Phosphate-Buffered Saline) which yields a suspension that is both isotonic and has a physiologically relevant pH.

In order to determine whether the generated particles have reduced interactions with mucus and are therefore able to move within mucus without becoming trapped, particles are incubated with human cervicovaginal mucus (CVM) and observed via dark field microscopy. One microliter or less of the nanoparticle suspension is added to 20 μL of CVM. Observations are made in a minimum of three distinct and randomly selected areas of the CVM sample. Control particles with known behavior are used to qualify the CVM sample as appropriate for the assay. Mobility in mucus is observed and therefore the nanoparticles are deemed to be effective MPP.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A pharmaceutical composition, comprising:
a plurality of mucus-penetrating coated particles, each coated particle comprising:
a core particle comprising loteprednol etabonate crystalline form I or form II, and a mucus penetration-enhancing coating comprising a surface-altering agent surrounding the core particle,
wherein the surface-altering agent comprises one or more of the following components:
a) a triblock copolymer comprising a hydrophilic block—hydrophobic block—hydrophilic block configuration, wherein the hydrophobic block has a molecular weight of at least about 2 kDa, and the hydrophilic blocks constitute at least about 15 wt % of the triblock copolymer, wherein the hydrophobic block associates with the surface of the core particle, and wherein the hydrophilic block is present at the surface of the coated particle and renders the coated particle hydrophilic,
b) a synthetic polymer having pendant hydroxyl groups on the backbone of the polymer, the polymer having a molecular weight of at least about 1 kDa and less than or equal to about 1000 kDa, wherein the polymer has a degree of hydrolysis of at least about 30% and less than about 95%, or
c) a polysorbate, and
at least one pharmaceutically acceptable carrier, additive, or diluent;
wherein the surface-altering agent is present on the outer surface of the core particle at a density of at least 0.01 molecules/nm²,
wherein the surface-altering agent is present in the pharmaceutical composition in an amount of between about 0.001% to about 5% by weight in total.

2. The pharmaceutical composition of claim 1, wherein the crystalline form I of loteprednol etabonate has X-ray powder diffraction (XRPD) peaks at about 5.6, 7.7, 11.9, 14.1, 17.0 and 18.8±0.2° 2θ; and wherein the crystalline form II of loteprednol etabonate has X-ray powder diffraction (XRPD) peaks at about 15.0°, 18.1°, and 19.8°±0.2° 2θ.

3. The pharmaceutical composition of claim 2, wherein the crystalline form I of loteprednol etabonate has further XRPD peaks at about 16.0, 21.0 and 22.0±0.2° 2θ; and wherein the crystalline form II of loteprednol etabonate has further XRPD peaks at about 9.8°, 15.6°, 16.6°, 17.2°, 23.0°, 24.8°, and 26.3°±0.2° 2θ.

4. The pharmaceutical composition of claim 1, wherein the surface-altering agent is present on the surfaces of the coated particles at a density of at least about 0.1 molecules per nanometer squared.

5. The pharmaceutical composition of claim 1, wherein the surface-altering agent is non-covalently adsorbed to the core particles.

6. The pharmaceutical composition of claim 1, wherein the surface-altering agent comprises the triblock copolymer.

7. The pharmaceutical composition of claim 6, wherein the triblock copolymer is poly(ethylene oxide)-poly(propylene oxide)-poly(ethylene oxide).

8. The pharmaceutical composition of claim 1, wherein the surface altering agent is poly(vinyl alcohol).

9. The pharmaceutical composition of claim 1, wherein the crystalline form I or form II of loteprednol etabonate comprises at least about 80 wt % of the core particle.

10. The pharmaceutical composition of claim 1, wherein the coated particles have an average size of about 10 nm to about 1 μm.

11. The pharmaceutical composition of claim 1, wherein the polydispersity index of the composition is less than or equal to about 0.5.

12. The pharmaceutical composition of claim 1, wherein the pharmaceutical composition is suitable for topical administration to the eye.

13. The pharmaceutical composition of claim 1, wherein the pharmaceutical composition is suitable for direct injection into the eye.

14. The pharmaceutical composition of claim 1, wherein the pharmaceutically acceptable carrier, additive, or diluent comprises glycerin.

15. The pharmaceutical composition of claim 1, wherein the composition comprises about 0.1 to about 1% w/v sodium chloride.

16. A method of treating, diagnosing, preventing, or managing an ocular condition in a subject, the method comprising: administering a pharmaceutical composition of claim 1 to an eye of a subject and thereby delivering the loteprednol etabonate, to a tissue in the eye of the subject.

17. The method of claim 16, comprising sustaining an ophthalmically efficacious level of the loteprednol etabonate, in a palpebral conjunctiva, a fornix conjunctiva, a bulbar conjunctiva, or a cornea for at least 12 hours after administration.

18. The method of claim 16, comprising delivering the loteprednol etabonate, to a tissue in the front of the eye of the subject or to tissue in the back of the eye of the subject.

19. The method of claim 16, wherein the ocular condition is inflammation, pain, macular degeneration, macular edema, uveitis, or dry eye.

20. A method of sustaining an ophthalmically efficacious level of loteprednol etabonate in an eye tissue in a subject in need thereof, comprising administering a pharmaceutical composition of claim 1 to the eye tissue of the subject, wherein the level of loteprednol etabonate is sustained for at least 12 hours after administration.
